(19) United States
(12) Patent Application Publication
(10) Pub. No.: US 2018/0263501 A1
(43) Pub. Date: Sep. 20, 2018

(54) ELECTRONIC DEVICE AND CONTROL METHOD THEREFOR

(71) Applicant: LG ELECTRONICS INC., Seoul (KR)

(72) Inventors: Hongjo SHIM, Seoul (KR); Hyunwoo KIM, Seoul (KR); Gukchan LIM, Seoul (KR); Yoonwoo LEE, Seoul (KR); Seonghyok KIM, Seoul (KR); Minhyun PARK, Seoul (KR)

(21) Appl. No.: 15/744,028

(22) PCT Filed: Sep. 1, 2015
(86) PCT No.: PCT/KR2015/009189

§ 371 Date: Jan. 11, 2018

(30) Foreign Application Priority Data
Jul. 15, 2015 (KR) 10-2015-0100270

(51) Int. Cl. A61B 5/01; A61B 5/00

(52) U.S. Cl. CPC A61B 5/01; A61B 5/441; A61B 2562/0271; A61B 5/6831; A61B 5/6824

(57) ABSTRACT

An electronic device and a method for controlling the same are disclosed. The electronic device includes a main body having a back surface contacting a body part of a user, a band portion extended from one side or both sides of the main body, the band portion having a back surface contacting the body part of the user, an electrode positioned on the back surface of the main body or the back surface of the band portion, the electrode having a current flow area and a non-current flow area, and a controller mounted inside the main body or the band portion, the controller configured to adjust a voltage applied to the electrode in response to a biometric signal of the user acquired through the electrode.

### TECHNICAL FIELD

The present disclosure relates to an electronic device and a method for controlling the same, and more particularly to an electronic device and a method for controlling the same for efficiently obtaining biometric information of a user.

### BACKGROUND ART

Terminals may be generally classified into mobile/portable terminals and stationary terminals based on a mobility. The mobile terminals may also be classified into handheld terminals and vehicle mounted terminals depending on whether or not a user can directly carry the terminal.

Mobile terminals have increasingly more functions. Examples of the functions include data and voice communications, taking pictures and videos with a camera, recording audio, playing music files using a speaker system, and displaying images and video on a display. Some mobile terminals include additional functionality which supports game playing, while other terminals are configured as multimedia players. More recently, the mobile terminals have been configured to receive broadcast and multicast signals which permit viewing of content such as videos and television programs.

As the mobile terminals have increasingly more functions, the mobile terminals have been implemented as multimedia players of multiple functions having taking pictures and videos, playing music files or video, game playing, receiving broadcast, and the like.

Efforts are ongoing to support and increase the functionality of mobile terminals. Such efforts include software and hardware improvements, as well as changes and improvements in the structural components.

A study on wearable electronic devices the user wears is being recently carried out. For example, an attempt has been made to study glass wearable electronic devices, watch wearable electronic devices, and the like.

Because the wearable electronic device has to arrange necessary electronic components in a limited space while satisfying design requirements, a need for an optimum design of the wearable electronic device is increasing.

### DISCLOSURE

### Technical Problem

An object of the present disclosure is to address the above-described and other problems. Another object of the present disclosure is to efficiently obtain biometric information of a user.

Another object of the present disclosure is to improve quality of a biometric signal.

Another object of the present disclosure is to provide a heatable electrode.

Another object of the present disclosure is to adjust a voltage applied to an electrode.

Another object of the present disclosure is to provide a temperature controllable electrode depending on a skin temperature of a user.

Another object of the present disclosure is to prevent low temperature burns from occurring on a user's skin.

### Technical Solution

In one aspect of the present disclosure, there is provided an electronic device comprising a main body having a back surface contacting a body part of a user; a band portion extended from one side or both sides of the main body, the band portion having a back surface contacting the body part of the user; an electrode positioned on the back surface of the main body or the back surface of the band portion, the electrode having a current flow area and a non-current flow area; and a controller mounted inside the main body or the band portion, the controller configured to adjust a voltage applied to the electrode in response to a biometric signal of the user acquired through the electrode.

According to another aspect of the present disclosure, the electronic device may further comprise a driver configured to adjust the voltage applied to the electrode by the controller.

According to another aspect of the present disclosure, the non-current flow area may be formed by an empty vacancy.

According to another aspect of the present disclosure, the current flow area may have conductivity, and the non-current flow area may have non-conductivity.

According to another aspect of the present disclosure, the electrode may have a plurality of rows and a plurality of columns. The non-current flow area may be formed at intersections of the plurality of rows and the plurality of columns.

According to another aspect of the present disclosure, the electrode may have a plurality of rows and a plurality of columns. The current flow area may be formed at intersections of the plurality of rows and the plurality of columns.

According to another aspect of the present disclosure, the electrode may have a conductive path connecting the intersections to one another.

According to another aspect of the present disclosure, the electrode may have a plurality of rows and a plurality of columns. The non-current flow area may be formed at intersections of odd-numbered rows of the plurality of rows and odd-numbered columns of the plurality of columns.

According to another aspect of the present disclosure, the electrode may have a plurality of rows and a plurality of columns. The non-current flow area may be formed at intersections of even-numbered rows of the plurality of rows and even-numbered columns of the plurality of columns.

According to another aspect of the present disclosure, the electrode may have a plurality of rows and a plurality of columns. The current flow area may be formed at intersections of odd-numbered rows of the plurality of rows and odd-numbered columns of the plurality of columns.

According to another aspect of the present disclosure, the electrode may have a plurality of rows and a plurality of columns. The current flow area may be formed at intersections of even-numbered rows of the plurality of rows and even-numbered columns of the plurality of columns.

According to another aspect of the present disclosure, the electrode may have a conductive path connecting the intersections to one another.

According to another aspect of the present disclosure, the electrode may include a heating element, and the controller may adjust a temperature of the heating element.

According to another aspect of the present disclosure, the band portion may be extended from both sides of the main body to wrap the body part of the user. The electrode may include a plurality of electrodes. At least one of the plurality of electrodes may be positioned on the back surface of the band portion extended from one side of the main body, and at least another of the plurality of electrodes may be positioned on the back surface of the band portion extended from the other side of the main body.

According to another aspect of the present disclosure, the electronic device may further comprise a sensor configured to measure a skin temperature of the user. The controller may adjust the voltage applied to the electrode depending on the skin temperature of the user obtained by the sensor.

According to another aspect of the present disclosure, the controller may reduce the voltage applied to the electrode when the skin temperature of the user exceeds a predetermined temperature.

In another aspect of the present disclosure, there is provided a method for controlling an electronic device comprising measuring a quality of a biometric signal of a user acquired by an electrode, analyzing whether or not biometric information of the user is obtained from the biometric signal, increasing a voltage applied to the electrode according to the analysis result, and measuring a quality of a biometric signal of the user acquired by the electrode of which the voltage is increased.

According to another aspect of the present disclosure, the method may further comprise, when the quality of the biometric signal is good, displaying the biometric information of the user.

According to another aspect of the present disclosure, the method may further comprise measuring a skin temperature of the user when the voltage applied to the electrode is increased, and reducing the voltage applied to the electrode when the skin temperature of the user exceeds a predetermined level.

According to another aspect of the present disclosure, the method may further comprise, when the quality of the biometric signal is low, displaying a notification of wear of the electronic device.

### Advantageous Effects

According to at least one aspect of the present disclosure, the present disclosure can efficiently obtain biometric information of a user.

According to at least one aspect of the present disclosure, the present disclosure can improve quality of a biometric signal.

According to at least one aspect of the present disclosure, the present disclosure can provide a heatable electrode.

According to at least one aspect of the present disclosure, the present disclosure can adjust a voltage applied to an electrode.

According to at least one aspect of the present disclosure, the present disclosure can provide a temperature controllable electrode depending on a skin temperature of a user.

According to at least one aspect of the present disclosure, the present disclosure can prevent low temperature burns from occurring on a user's skin.

### DESCRIPTION OF DRAWINGS

FIG. 1 is a block diagram of an electronic device related to an embodiment of the disclosure.

FIG. 2 is a perspective view of an electronic device related to an embodiment of the disclosure.

FIG. 3 is an exploded perspective view of an electronic device shown in FIG. 2.

FIGS. 4 and 5 are plan views illustrating a band substrate of an electronic device related to an embodiment of the disclosure.

FIG. 6 illustrates another example of an electronic device related to an embodiment of the disclosure.

FIG. 7 illustrates an example of an electronic device according to an embodiment of the disclosure.

FIG. 8 illustrates an example of a band portion according to an embodiment of the disclosure.

FIG. 9 illustrates an example of a cross section of an electronic device according to an embodiment of the disclosure.

FIG. 10 illustrates an example of a flexible printed circuit board and electrodes according to an embodiment of the disclosure.

FIG. 11 illustrates an example of an electrode according to an embodiment of the disclosure.

FIGS. 12 to 43 illustrate various examples of an electrode according to an embodiment of the disclosure.

FIG. 44 illustrates an example of a temperature distribution of an electrode according to an embodiment of the disclosure.

FIG. 45 illustrates an example of a cross section of an electrode according to an embodiment of the disclosure.

FIG. 46 illustrates another example of a cross section of an electrode according to an embodiment of the disclosure.

FIGS. 47 to 50 illustrate examples of a biometric signal measured using an electrode.

FIG. 51 illustrates an example of a skin temperature measured in a state where a skin contacts an electrode according to an embodiment of the disclosure.

FIG. 52 illustrates an example of a method for controlling an electronic device according to an embodiment of the disclosure.

FIG. 53 illustrates another example of a method for controlling an electronic device according to an embodiment of the disclosure.

FIG. 54 illustrates an example of a method for operating an electronic device according to an embodiment of the disclosure.

FIG. 55 illustrates another example of a method for operating an electronic device according to an embodiment of the disclosure.

FIG. 56 illustrates yet another example of a method for operating an electronic device according to an embodiment of the disclosure.

### MODE FOR INVENTION

Reference will now be made in detail to embodiments of the disclosure, examples of which are illustrated in the accompanying drawings. Wherever possible, the same reference numbers will be used throughout the drawings to refer to the same or like parts. In general, a suffix such as “module” and “unit” may be used to refer to elements or components. Use of such a suffix herein is merely intended to facilitate description of the specification, and the suffix itself is not intended to give any special meaning or function. It will be noted that a detailed description of known arts will be omitted if it is determined that the detailed description of the known arts can obscure the embodiments of the disclosure. The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings.

The terms ‘first’, ‘second’, etc. may be used to describe various components, but the components are not limited by such terms. The terms are used only for the purpose of distinguishing one component from other components.

When an arbitrary component is described as “being connected to” or “being coupled to” another component, this should be understood to mean that still another component(s) may exist between them, although the arbitrary component may be directly connected to or directly coupled to another component. In contrast, when an arbitrary component is described as “being directly connected to” or “being directly coupled to” another component, this should be understood to mean that no component exists between them.

A singular expression can include a plural expression as long as it does not have an apparently different meaning in context.

In the present application, the terms “include” and “have” should be understood to be intended to designate that illustrated features, numbers, steps, operations, components, parts or combinations thereof exist and not to preclude the existence of one or more different features, numbers, steps, operations, components, parts or combinations thereof, or the possibility of the addition thereof.

Electronic devices disclosed herein may be implemented using a variety of different types of devices. Examples of such devices include cellular phones, smart phones, laptop computers, digital broadcast terminals, personal digital assistants (PDAs), portable multimedia players (PMPs), navigators, slate computers (PCs), tablet PCs, ultra books, wearable devices (for example, smart watches, smart glasses, head mounted displays (HMDs)), and the like.

By way of non-limiting example only, further description will be made with reference to particular types of electronic devices. However, such teachings may be equally applied to other types of electronic devices, such as those types noted above. In addition, these teachings may also be applied to stationary terminals such as digital TV, desktop computers, digital signage, and the like.

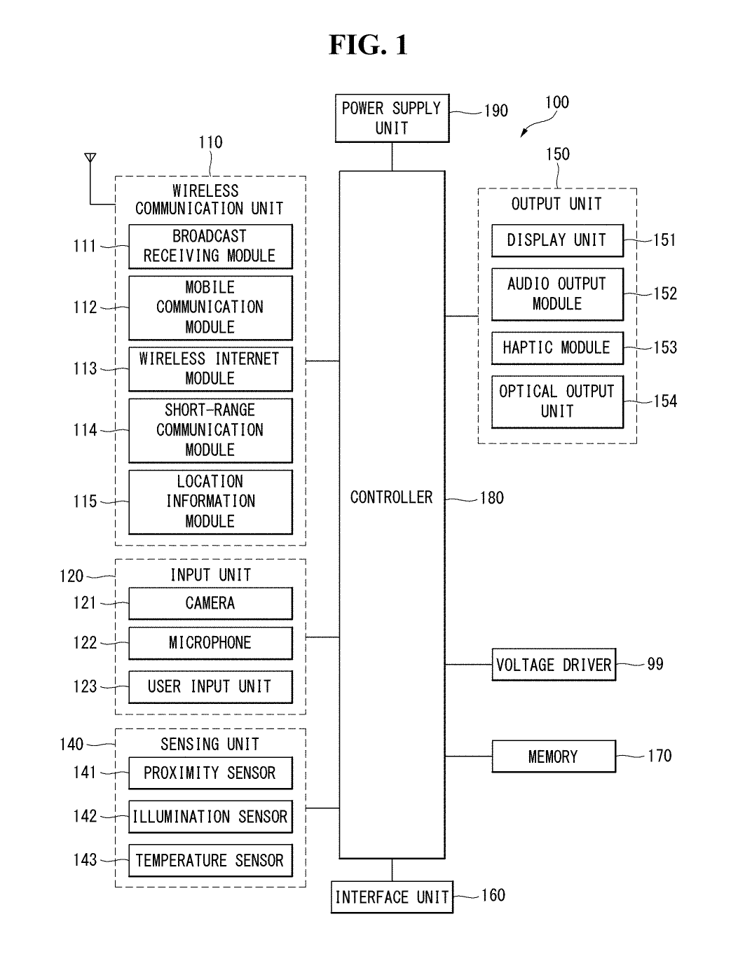

FIG. 1 is a block diagram of an electronic device related to an embodiment of the disclosure.

An electronic device 100 may include a voltage driver 99, a wireless communication unit 110, an input unit 120, a sensing unit 140, an output unit 150, an interface unit 160, a memory 170, a controller 180, a power supply unit 190, and the like. It is understood that implementing all of the components illustrated in FIG. 1 is not a requirement for the electronic device, and that more or fewer components may be alternatively implemented.

More specifically, the voltage driver 99 can adjust a voltage applied to an electrode 700 which will be described later. The voltage driver 99 may generate various voltages applied to the electrode 700. The generation of the various voltages of the voltage driver 99 may be controlled by the controller 180 which will be described later.

The wireless communication unit 110 may include one or more modules which permit communications such as wireless communications between the electronic device 100 and a wireless communication system, communications between the electronic device 100 and another electronic device, and communications between the electronic device 100 and an external server. Further, the wireless communication unit 110 may include one or more modules which connect the electronic device 100 to one or more networks.

The wireless communication unit 110 may include one or more of a broadcast receiving module 111, a mobile communication module 112, a wireless Internet module 113, a short-range communication module 114, and a location information module 115.

The input unit 120 may include a camera 121 which is one type of an image input unit for obtaining images or video, a microphone 122 which is one type of an audio input unit for inputting an audio signal, a user input unit 123 (for example, a touch key, a push key, a mechanical key, and the like) for allowing a user to input information, and the like. Data (for example, audio, video, image, and the like) obtained by the input unit 120 may be analyzed and processed by user control commands.

The sensing unit 140 may be implemented using one or more sensors configured to sense internal information of the electronic device, information about a surrounding environment of the electronic device, user information, and the like. For example, the sensing unit 140 may include one or more of a proximity sensor 141, an illumination sensor 142, a touch sensor, an acceleration sensor, a magnetic sensor, a G-sensor, a gyroscope sensor, a motion sensor, an RGB sensor, an infrared (IR) sensor, a fingerprint scan sensor, a ultrasonic sensor, an optical sensor (for example, camera 121), a microphone 122, a battery gauge, an environment sensor (for example, a barometer, a hygrometer, a thermometer, a radiation detection sensor, a thermal sensor, and a gas sensor, and the like), and a chemical sensor (for example, an electronic nose, a health care sensor, a biometric sensor, and the like). The electronic device 100 according to the embodiment of the disclosure may be configured to utilize information obtained from the sensing unit 140, and in particular, information obtained from two or more sensors of the sensing unit 140, and combinations thereof. Other embodiments, the environment sensor may include a temperature sensor 143.

The output unit 150 may be typically configured to output various types of information, such as audio, video, tactile output, and the like. The output unit 150 may include one or more of a display unit 151, an audio output module 152, a haptic module 153, and an optical output unit 154. The display unit 151 may have an inter-layered structure or an integrated structure with a touch sensor in order to implement a touch screen. The touch screen may provide an output interface between the electronic device 100 and the user, as well as function as the user input unit 123 which provides an input interface between the electronic device 100 and the user.

The interface unit 160 serves as an interface with various types of external devices that can be coupled to the electronic device 100. The interface unit 160 may include one or more of wired or wireless headset ports, external power supply ports, wired or wireless data ports, memory card ports, ports for connecting a device having an identification module, audio input/output (I/O) ports, video I/O ports, and earphone ports. In some cases, the electronic device 100 may perform assorted control functions associated with a connected external device, in response to the external device being connected to the interface unit 160.

The memory 170 is typically implemented to store data to support various functions or features of the electronic device 100. For instance, the memory 170 may be configured to store application programs executed in the electronic device 100, data or instructions for operations of the electronic device 100, and the like. Some of these application programs may be downloaded from an external server via wireless communication. Other application programs may be installed within the electronic device 100 at time of manufacturing or shipping, which is typically the case for basic functions (for example, receiving a call, placing a call, receiving a message, sending a message, and the like) of the electronic device 100. It is common for application programs to be stored in the memory 170, installed in the electronic device 100, and executed by the controller 180 to perform an operation (or function) for the electronic device 100.

The controller 180 typically functions to control overall operation of the electronic device 100, in addition to the operations associated with the application programs. The controller 180 may provide or process information or functions appropriate for a user by processing signals, data, information and the like, which are input or output by the various components depicted in FIG. 1, or activating application programs stored in the memory 170. The controller 180 may control some or all of the components illustrated in FIG. 1 according to the execution of an application program that have been stored in the memory 170. In addition, the controller 180 may combine and operate at least two of the components included in the electronic device 100 for the execution of the application program.

The power supply unit 190 may be configured to receive external power or provide internal power and supply appropriate power required for operating the components included in the electronic device 100 under the control of the controller 180. The power supply unit 190 may include a battery, and the battery may be configured to be embedded in the device body, or configured to be detachable from the device body.

At least some of the above components may be combined with one another and operate, in order to implement an operation, a control, or a control method of an electronic device according to various embodiments described below. Further, an operation, a control, or a control method of an electronic device according to various embodiments may be implemented by an execution of at least one application program stored in the memory 170.

Before describing various embodiments implemented by the above-described electronic device 100, various components depicted in this figure will now be described in more detail with reference to FIG. 1.

Regarding the wireless communication unit 110, the broadcast receiving module 111 is typically configured to receive a broadcast signal and/or broadcast associated information from an external broadcast managing entity via a broadcast channel. The broadcast channel may include a satellite channel, a terrestrial channel, etc. In some embodiments, two or more broadcast receiving modules 111 may be utilized to facilitate simultaneously receiving of two or more broadcast channels, or to support switching among broadcast channels.

The mobile communication module 112 can transmit and/or receive wireless signals to and from one or more network entities. Examples of the network entity include a base station, an external electronic device, a server, and the like. Such network entities form part of a mobile communication network, which is constructed according to technical standards or communication methods for mobile communications (for example, Global System for Mobile Communication (GSM), Code Division Multi Access (CDMA), CDMA2000 (Code Division Multi Access 2000), EV-DO (Enhanced Voice-Data Optimized or Enhanced Voice-Data Only), Wideband CDMA (WCDMA), High Speed Downlink Packet access (HSDPA), HSUPA (High Speed Uplink Packet Access), Long Term Evolution (LTE), LTE-A (Long Term Evolution-Advanced), and the like).

Examples of the wireless signals transmitted and/or received via the mobile communication module 112 include audio call signals, video (telephony) call signals, or various formats of data to support communication of text and multimedia messages.

The wireless Internet module 113 is configured to facilitate wireless Internet access. The wireless Internet module 113 may be internally or externally coupled to the electronic device 100. The wireless Internet module 113 may transmit and/or receive wireless signals via communication networks according to wireless Internet technologies.

Examples of the wireless Internet technology include Wireless LAN (WLAN), Wireless Fidelity (Wi-Fi), Wi-Fi Direct, Digital Living Network Alliance (DLNA), Wireless Broadband (WiBro), Worldwide Interoperability for Microwave Access (WiMAX), High Speed Downlink Packet Access (HSDPA), HSUPA (High Speed Uplink Packet Access), Long Term Evolution (LTE), LTE-A (Long Term Evolution-Advanced), and the like. The wireless Internet module 113 may transmit/receive data according to one or more of such wireless Internet technologies, and other Internet technologies as well.

In some embodiments, when the wireless Internet access is implemented according to, for example, WiBro, HSDPA, HSUPA, GSM, CDMA, WCDMA, LTE, LTE-A and the like, as part of a mobile communication network, the wireless Internet module 113 performs such wireless Internet access. As such, the Internet module 113 may cooperate with, or function as, the mobile communication module 112.

The short-range communication module 114 is configured to facilitate short-range communications. Suitable technologies for implementing such short-range communications include Bluetooth™, Radio Frequency IDentification (RFID), Infrared Data Association (IrDA), Ultra-WideBand (UWB), ZigBee, Near Field Communication (NFC), Wireless-Fidelity (Wi-Fi), Wi-Fi Direct, Wireless USB (Wireless Universal Serial Bus), and the like. The short-range communication module 114 generally supports wireless communications between the electronic device 100 and a wireless communication system, communications between the electronic device 100 and another electronic device 100, or communications between the electronic device and a network where another electronic device 100 (or an external server) is located, via wireless area networks. One example of the wireless area networks may be a wireless personal area network.

In some embodiments, another electronic device (which may be configured similarly to the electronic device 100) may be a wearable device, for example, a smart watch, a smart glass or a head mounted display (HIVID), which is able to exchange data with the electronic device 100 (or otherwise cooperate with the electronic device 100). The short-range communication module 114 may sense or recognize the wearable device, and permit communication between the wearable device and the electronic device 100. In addition, when the sensed wearable device is a device which is authenticated to communicate with the electronic device 100, the controller 180 may cause transmission of data processed in the electronic device 100 to the wearable device via the short-range communication module 114. Hence, a user of the wearable device may use the data processed in the electronic device 100 on the wearable device. For example, when a call is received in the electronic device 100, the user may answer the call using the wearable device. Also, when a message is received in the electronic device 100, the user can check the received message using the wearable device.

The location information module 115 is generally configured to detect, calculate, derive or otherwise identify a position of the electronic device. As an example, the location information module 115 includes a Global Position System (GPS) module, a Wi-Fi module, or etc. As one example, when the electronic device uses a GPS module, a position of the electronic device may be acquired using a signal sent from a GPS satellite. As another example, when the electronic device uses the Wi-Fi module, a position of the electronic device can be acquired based on information related to a wireless access point (AP) which transmits or receives a wireless signal to or from the Wi-Fi module. If desired, the location information module 115 may alternatively or additionally function with any of the other modules of the wireless communication unit 110 to obtain data related to the position of the electronic device. The location information module 115 is a module used to obtain a position (or current position) of the electronic device, and is not limited to a module that directly calculates or obtains the position of the electronic device

The input unit 120 may be configured to permit various types of input to the electronic device 100. Examples of such input include audio, image, video, data, and user input. Image and video input is often obtained using one or more cameras 121. Such cameras 121 may process image frames of still pictures or video obtained by image sensors in a video or image capture mode. The processed image frames can be displayed on the display unit 151 or stored in memory 170. In some cases, the cameras 121 may be arranged in a matrix configuration to permit a plurality of images having various angles or focal points to be input to the electronic device 100. As another example, the cameras 121 may be located in a stereoscopic arrangement to acquire left and right images for implementing a stereoscopic image.

The microphone 122 is generally implemented to permit audio input to the electronic device 100. The audio input can be processed in various manners in accordance with a function being executed in the electronic device 100. If desired, the microphone 122 may include assorted noise removing algorithms to remove unwanted noise generated in the course of receiving the external audio.

The user input unit 123 is a component that permits input by a user. Such user input may enable the controller 180 to control operation of the electronic device 100. The user input unit 123 may include one or more of a mechanical input element (for example, a key, a button located on a front and/or rear surface or a side surface of the electronic device 100, a dome switch, a jog wheel, a jog switch, and the like), or a touch-sensitive input, among others. As one example, the touch-sensitive input may be a virtual key or a soft key, which is displayed on a touch screen through software processing, or a touch key which is located on the electronic device at a location that is other than the touch screen. On the other hand, the virtual key or the visual key may be displayed on the touch screen in various shapes, for example, graphic, text, icon, video, or a combination thereof.

The sensing unit 140 is generally configured to sense one or more of internal information of the electronic device, surrounding environment information of the electronic device, user information, or the like, and generate a sensing signal corresponding to the information. The controller 180 generally cooperates with the sending unit 140 to control operation of the electronic device 100 or execute data processing, a function or an operation associated with an application program installed in the electronic device 100 based on the sensing signal provided by the sensing unit 140. The sensing unit 140 may be implemented using any of a variety of sensors, some of which will now be described in more detail.

The proximity sensor 141 may include a sensor to sense presence or absence of an object approaching a predetermined surface, or an object located near a surface, by using an electromagnetic field, infrared rays, or the like without a mechanical contact. The proximity sensor 141 may be arranged at an inner region of the electronic device 100 covered by the touch screen, or near the touch screen.

The proximity sensor 141, for example, may include any of a transmissive photoelectric sensor, a direct reflective photoelectric sensor, a mirror reflective photoelectric sensor, a high-frequency oscillation proximity sensor, a capacitive proximity sensor, a magnetic proximity sensor, an infrared proximity sensor, and the like. When the touch screen is implemented as a capacitive touch sensor, the proximity sensor 141 can sense proximity of a pointer relative to the touch screen by changes of an electromagnetic field, which is responsive to an approach of an object with conductivity. In this instance, the touch screen (or the touch sensor) may also be categorized as a proximity sensor.

The term “proximity touch” will often be referred to herein to denote the scenario in which a pointer is positioned to be proximate to the touch screen without contacting the touch screen. The term “contact touch” will often be referred to herein to denote the scenario in which a pointer makes physical contact with the touch screen. For the position corresponding to the proximity touch of the pointer relative to the touch screen, such position will correspond to a position where the pointer is perpendicular to the touch screen. The proximity sensor 141 may sense proximity touch, and proximity touch patterns (for example, a proximity touch distance, a proximity touch direction, a proximity touch speed, a proximity touch time, a proximity touch position, a proximity touch moving status, and the like). In general, controller 180 processes data corresponding to proximity touches and proximity touch patterns sensed by the proximity sensor 141, and cause output of visual information on the touch screen. In addition, the controller 180 can control the electronic device 100 to execute different operations or process different data according to whether a touch with respect to a point on the touch screen is either a proximity touch or a contact touch.

A touch sensor can sense a touch applied to the touch screen, such as display unit 151, using any of a variety of touch methods. Examples of such touch methods include a resistive type, a capacitive type, an infrared type, and a magnetic field type, among others.

As one example, the touch sensor may be configured to convert changes of pressure applied to a specific part of the touch sensor or capacitance occurring at a specific part of the touch sensor into electric input signals. The touch sensor may also be configured to sense not only a touched position and a touched area of the touch screen, but also a touch pressure and/or a touch capacitance at a touch operation. A touch object is generally used to apply a touch input to the touch sensor. Examples of the touch object include a finger, a touch pen, a stylus pen, a pointer, or the like.

When a touch input is sensed by a touch sensor, signals corresponding to the touch input may be transmitted to a touch controller. The touch controller may process the received signals and then transmit corresponding data to the controller 180. Thus, the controller 180 may sense which region of the display unit 151 has been touched. In embodiments disclosed herein, the touch controller may be a component separate from the controller 180, the controller 180, and combinations thereof.

In some embodiments, the controller 180 may execute the same control or different controls in accordance with a kind of a touch object that touches the touch screen (or a touch key provided in addition to the touch screen). Whether to execute the same control or different controls in accordance with the touch object which provides a touch input may be determined based on a current operating state of the electronic device 100 or a currently executed application program, for example.

The touch sensor and the proximity sensor may be implemented individually, or in combination, to sense various types of touches. Such touches includes a short (or tap) touch, a long touch, a multi-touch, a drag touch, a flick touch, a pinch-in touch, a pinch-out touch, a swipe touch, a hovering touch, and the like.

If desired, an ultrasonic sensor may be implemented to recognize position information relating to a touch object using ultrasonic waves. The controller 180, for example, may calculate a position of a wave generation source based on information sensed by an optical sensor and a plurality of ultrasonic sensors. Since light is much faster than ultrasonic waves, the time for which the light reaches the optical sensor is much shorter than the time for which the ultrasonic wave reaches the ultrasonic sensor. The position of the wave generation source may be calculated using this fact. For instance, the position of the wave generation source may be calculated using the time difference from the time that the ultrasonic wave reaches the sensor based on the light as a reference signal.

The camera 121 typically includes at least one a camera sensor (CCD, CMOS etc.), a photo sensor (or image sensors), and a laser sensor.

Implementing the camera 121 with a laser sensor may allow detection of a touch of a physical object with respect to a 3D stereoscopic image. The photo sensor may be laminated on or overlapped with the display device. The photo sensor may be configured to scan movement of the physical object in proximity to the touch screen. In more detail, the photo sensor may include photo diodes and transistors at rows and columns to scan content received at the photo sensor using an electrical signal which changes according to the quantity of applied light. Namely, the photo sensor may calculate the coordinates of the physical object depending on variation of light to thus obtain position information of the physical object.

The display unit 151 is generally configured to output information processed in the electronic device 100. For example, the display unit 151 may display execution screen information of an application program executing at the electronic device 100 or user interface (UI) and graphic user interface (GUI) information in response to the execution screen information.

In some embodiments, the display unit 151 may be implemented as a stereoscopic display unit for displaying stereoscopic images.

The stereoscopic display unit may employ a 3D display scheme such as a stereoscopic scheme (a glass scheme), an auto-stereoscopic scheme (glassless scheme), a projection scheme (holographic scheme), or the like.

The audio output module 152 is generally configured to output audio data. Such audio data may be obtained from any of a number of different sources, such that the audio data may be received from the wireless communication unit 110 or may have been stored in the memory 170. The audio data may be output during modes such as a signal reception mode, a call mode, a record mode, a voice recognition mode, a broadcast reception mode, and the like. The audio output module 152 can provide audible output related to a particular function (e.g., a call signal reception sound, a message reception sound, etc.) performed by the electronic device 100. The audio output module 152 may also be implemented as a receiver, a speaker, a buzzer, or the like.

The haptic module 153 is configured to generate various tactile effects that a user feels, perceive, or otherwise experience. A typical example of a tactile effect generated by the haptic module 153 is a vibration. A strength, a pattern, and the like of the vibration generated by the haptic module 153 can be controlled by user selection or setting by the controller 180. For example, the haptic module 153 may output different vibrations in a combining manner or a sequential manner.

In addition to the vibration, the haptic module 153 can generate various other tactile effects, including an effect by stimulation such as a pin arrangement vertically moving to contact skin, a spray force or suction force of air through a jet orifice or a suction opening, a touch to the skin, a contact of an electrode, electrostatic force, an effect by reproducing the sense of cold and warmth using an element that can absorb or generate heat, and the like.

The haptic module 153 can also be implemented to allow the user to feel a tactile effect through a muscle sensation such as the user's fingers or arm, as well as transferring the tactile effect through direct contact. Two or more haptic modules 153 may be provided according to the particular configuration of the electronic device 100.

The optical output unit 154 outputs a signal for indicating an event generation using light of a light source. Examples of events generated in the electronic device 100 may include message reception, call signal reception, a missed call, an alarm, a schedule notice, an email reception, information reception through an application, and the like.

A signal output by the optical output unit 154 may be implemented in such a manner that the electronic device 100 emits monochromatic light or light with a plurality of colors to a front surface or a rear surface. The signal output may be terminated as the electronic device senses that a user has checked the generated event, for example.

The interface unit 160 serves as an interface for external devices to be connected with the electronic device 100. The interface unit 160 can receive data transmitted from an external device, receive power to transfer to elements and components within the electronic device 100, or transmit internal data of the electronic device 100 to such external device. For example, the interface unit 160 may include wired or wireless headset ports, external power supply ports, wired or wireless data ports, memory card ports, ports for connecting a device having an identification module, audio input/output (I/O) ports, video I/O ports, earphone ports, or the like.

The identification module may be a chip that stores various information for authenticating authority of using the electronic device 100 and may include a user identity module (UIM), a subscriber identity module (SIM), a universal subscriber identity module (USIM), and the like. In addition, the device having the identification module (also referred to herein as an “identifying device”) may be manufactured as a smart card. Thus, the identifying device can be connected with the electronic device 100 via the interface unit 160.

When the electronic device 100 is connected with an external cradle, the interface unit 160 can serve as a passage to allow power from the cradle to be supplied to the electronic device 100 or may serve as a passage to allow various command signals input by the user from the cradle to be transferred to the electronic device there through. Various command signals or power input from the cradle may operate as signals for recognizing that the electronic device is properly mounted on the cradle.

The memory 170 can store programs to support operations of the controller 180 and store input/output data (for example, phonebook, messages, still images, videos, etc.). The memory 170 may store data related to various patterns of vibrations and audio which are output in response to touch inputs on the touch screen.

The memory 170 may include one or more types of storage mediums including a Flash memory, a hard disk, a solid state disk, a silicon disk, a multimedia card micro type, a card-type memory (e.g., SD or DX memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, an optical disk, and the like. The electronic device 100 may also be operated in relation to a network storage device that performs the storage function of the memory 170 over a network, such as the Internet.

The controller 180 may typically control the general operations of the electronic device 100. For example, the controller 180 may set or release a lock state for restricting a user from inputting a control command with respect to applications when a status of the electronic device meets a preset condition.

The controller 180 can also perform the controlling and processing associated with voice calls, data communications, video calls, and the like, or perform pattern recognition processing to recognize a handwriting input or a picture drawing input performed on the touch screen as characters or images, respectively. In addition, the controller 180 can control one or a combination of those components in order to implement various embodiments disclosed herein.

The power supply unit 190 may receive external power or provide internal power and supply the appropriate power required for operating respective elements and components included in the electronic device 100. The power supply unit 190 may include a battery, which is typically rechargeable or be detachably coupled to the device body for charging.

The power supply unit 190 may include a connection port. The connection port may be configured as one example of the interface unit 160 to which an external charger for supplying power to recharge the battery is electrically connected.

As another example, the power supply unit 190 may be configured to recharge the battery in a wireless manner without use of the connection port. In this example, the power supply unit 190 can receive power, transferred from an external wireless power transmitter, using at least one of an inductive coupling method which is based on magnetic induction or a magnetic resonance coupling method which is based on electromagnetic resonance.

Various embodiments described herein may be implemented in a computer-readable medium, a machine-readable medium, or similar medium using, for example, software, hardware, or any combination thereof.

The electronic device may be expanded to a wearable device the user can directly wear beyond a hand-held device, which the user has and uses in his or her hand. Examples of the wearable device include a smart watch, a smart glass, and a head mounted display (HMD).

Examples of the electronic device expanded to the wearable device will now be described in more detail.

The wearable device may be configured to exchange (or interwork) data with another electronic device 100. The short-range communication module 114 may sense (or recognize) the wearable device, which is positioned around the electronic device 100 and can communicate with the electronic device 100. Furthermore, when the sensed wearable device is a device which is authenticated to communicate with the electronic device 100, the controller 180 may transmit at least a portion of data processed in the electronic device 100 to the wearable device via the short-range communication module 114. Thus, the user of the wearable device may use the data processed in the electronic device 100 on the wearable device. For example, when a call is received in the electronic device 100, the user may answer the call using the wearable device. Also, when a message is received in the electronic device 100, the user may check the received message using the wearable device.

At least some of the components illustrated in FIG. 1 may cooperatively operate to implement an operation, a control, or a control method of the electronic device 100 according to various embodiments of the disclosure that will be described below. The operation, the control, or the control method of the electronic device 100 may be implemented by the execution of at least one application program stored in the memory 170.

The watch electronic device 100 according to the embodiment of the disclosure is a kind of the mobile terminal which the user wears on his/her wrist. The watch electronic device 100 may include some or all of the components illustrated in FIG. 1. The characteristics of the watch electronic device 100 related to its shape will now be described in detail.

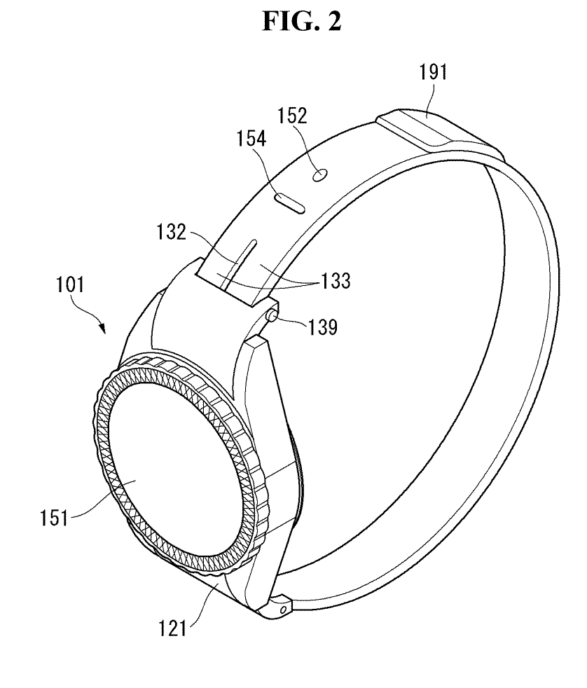

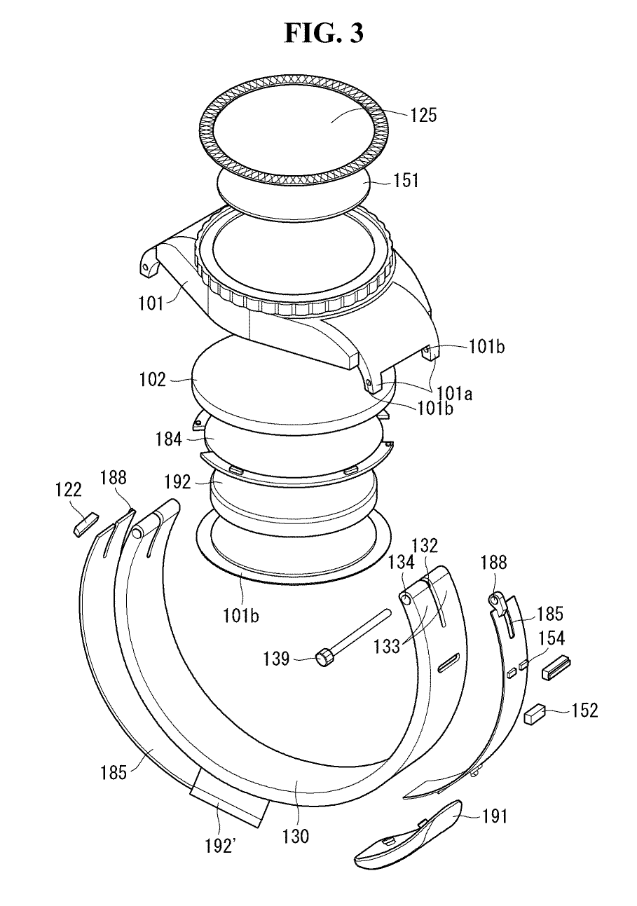

FIG. 2 is a perspective view of an electronic device related to an embodiment of the disclosure. FIG. 3 is an exploded perspective view of the electronic device shown in FIG. 2.

An electronic device according to an embodiment of the disclosure includes a band portion 130 which has a curved surface in a longitudinal direction or includes a flexible material. The band portion 130 may be detachable from a main body 101 of the electronic device using a hinge pin 139.

When the band portion 130 is made of a material with rigidity, the band portion 130 may have a curved shape. Alternatively, when the band portion 130 is made of the flexible material, the band portion 130 may be flexible. Hence, the user can wear the band portion 130 by winding the band portion 130 on his/her wrist. A mounting part, on which electronic components can be mounted, is provided inside the band portion 130. A band substrate 185, the audio output module 152, the microphone 122, the optical output unit 154, an antenna (not shown), etc. may be mounted on the mounting part.

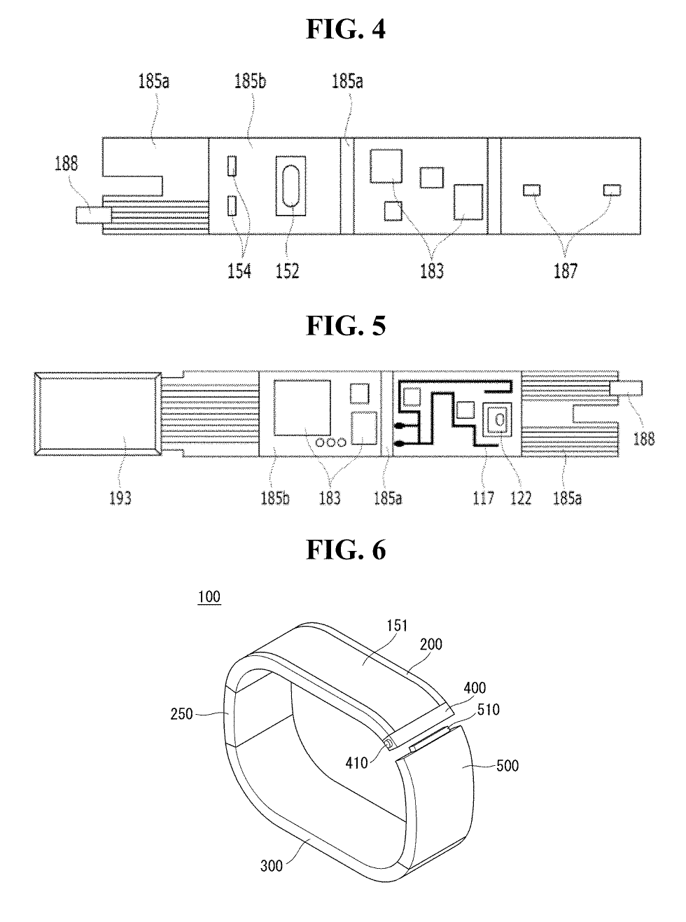

FIGS. 4 and 5 are plan views illustrating the band substrate 185 of the electronic device related to an embodiment of the disclosure. The band substrate 185 includes a flexible substrate. As shown in FIGS. 4 and 5, a substrate formed of a hard material may be configured as a plurality of parts, and the flexible substrate may be interposed between the plurality of parts. Alternatively, the band substrate 185 may be entirely formed of a flexible material.

An integrated circuit (IC) 183 is mounted on the band substrate 185 and controls the audio output module 152, the microphone 122, the optical output unit 154, the wireless communication unit 110, etc. mounted on the band portion 130. When the IC 183 is connected to the main body 101, the IC 183 may also control the main body 101. The audio output module 152, the microphone 122, the optical output unit 154, an antenna 117, etc. may be mounted on the band portion 130 separately from the band substrate 185, but may be mounted on the band substrate 185 as shown in FIGS. 4 and 5.

As shown in FIG. 3, the band substrates 185 respectively positioned on both sides of the band portion 130 may be separated from each other and may be combined to form one band substrate 185. Even if the band substrates 185 are separated from each other, the separated band substrates 185 may be connected to each other when ends of the band portion 130 are connected to the main body 101 or the ends of the band portion 130 are connected to each other.

The audio output module 152, the optical output unit 154, and the IC 183 are positioned on the band substrate 185 disposed on one side of the band portion 130. Also, a terminal connected to an external battery 191 may be positioned thereon. The microphone 122, the antenna 117, the IC 183, and an internal battery 192′ may be mounted on the band substrate 185 disposed on the other side of the band portion 130. The above arrangement of the band substrate 185 may be changed, and more components including the components noted above may be mounted on the band substrate 185.

A slit 132 extending in a longitudinal direction of the band portion 130 is positioned at the end of the band portion 130. In the embodiment of the disclosure, the slits 132 are respectively formed at both ends of the band portion 130, and each end of the band portion 130 is divided into two division ends 133 by the slit 132. The number of division ends 133 increases depending on an increase in the number of slits 132.

Even when the band portion 130 is made of the material with rigidity, the division end 133 may be made of a flexible material. The division ends 133 may bend up and down in a thickness direction of the band portion 130 and also may bend in different directions.

The band portion 130 may include a fastening hole 134 extending at the end of the band portion 130, i.e., at the side of the division end 133 in a width direction of the band portion 130. The hinge pin 139 is fastened to the fastening hole 134, thereby connecting the band portion 130 to the main body 101. The main body 101 includes a hinge hole 101b, through which the hinge pin 139 passes.

The hinge pin 139 passing through the band portion 130 may be formed of a conductive material and may be electrically connected to a connection ring 188 which is positioned inside the fastening hole 134 and the hinge hole 101b. The connection ring 188 is a ring-shaped member which is positioned inside the fastening hole 134 of the band portion 130 and is formed of the conductive material. An end of the connection ring 188 may be connected to the band substrate 185 mounted on the band portion 130.

A clock plate 102 including markings, an hour hand, a minute hand, a second hand, etc. is positioned on a front surface of the main body 101. The main body 101 includes a band fastening portion 101a, which is coupled to the band portion 130 through the hinge pin 139, at each side of the main body 101. The band fastening portion 101a includes a pair of fastening protrusions, which are spaced apart from each other by a distance corresponding to a width of the band portion 130, and the hinge holes 101b formed in the fastening protrusions. As described above, the hinge pin 139 is inserted into the hinge holes 101b and fastens the band portion 130 to the main body 101.

The main body 101 may be a clock body having only a function of a general wristwatch. The general wristwatch has the band fastening portion 101a so as to replace a band of the wristwatch, and the band portion 130 can be replaced by inserting the hinge pin 139 into the hinge holes 101b of the band fastening portion 101a. Thus, the electronic device according to the embodiment of the disclosure may be fastened to the general main body 101.

Even in case of the main body 101, on which the electronic components are not separately mounted, the main body 101 may include a battery 192 for driving the clock plate 102. The battery 192 may be used only in a drive of the clock plate 102. The battery included in the band portion 130 itself may be used to drive the electronic components of the band portion 130 and used to drive the display unit 151 when the display unit 151 is additionally coupled to the main body 101.

Alternatively, as shown in FIG. 3, the main body 101, on the electronic components are mounted, may be used. The main body 101 includes the display unit 151, a circuit unit 184 for the control, and a main battery 192 for supplying electric power. As shown in FIG. 3, the structure, for example, the camera 121, which is not included in the electronic device, may be included in the main body 101.

When the display unit 151 is used as a display of the general wristwatch, the display unit 151 is maintained in a transparent state. Only when information is output through the display unit 151, the display unit 151 may be changed to an opaque or translucent display. A touch sensor 125 is positioned on a front surface of the display unit 151 and may simultaneously perform input and output operations.

When the electronic components are mounted on the main body 101, the connection ring 188 is positioned inside the hinge hole 101b for the electrical connection between the electronic components. Hence, the electronic components of the main body 101 may be connected to the circuit unit 184 inside the main body 101 through the connection ring 188. A function of the electronic device may be expanded through the connection between the main body 101 and the band portion 130.

For example, when the band portion 130 is connected to the main body 101 having only a display function, wireless communication with a base station, or a call or transmission and reception of data through short range communication can be performed using the antenna 117 included in the band portion 130. Also, audio information may be output via the audio output module 152 included in the band portion 130.

In addition to the connection between the band portion 130 and the main body 101 through the end of the band portion 130, the main body 101 may be connected to an external power source through the hinge pin 139 to receive electric power, or may be connected to an external terminal, for example, a computer.

The electronic device according to the embodiment of the disclosure may apply a short range communication technology, such as Bluetooth™, Radio Frequency Identification (RFID), Infrared Data Association (IrDA), Ultra Wideband (UWB), ZigBee, Near Field Communication (NFC), and Wireless Universal Serial Bus (USB).

An NFC module included in the electronic device supports contactless type near field communication between terminals at a distance of typically 10 cm or less. The NFC module may operate in one of a card mode, a reader mode, and a peer-to-peer (P2P) mode. The electronic device 100 may further include a security module storing card information, in order to operate the NFC module in the card mode. In embodiments disclosed herein, the security module may be physical media, such as universal integrated circuit card (UICC) (for example, subscriber identification module (SIM) or universal SIM (USIM)), secure micro SD, and a sticker, and may be logical media (for example, embedded secure element (SE) embedded in the electronic device. Data exchange based on single wire protocol (SWP) may be performed between the NFC module and the security module.

When the NFC module operates in the card mode, the electronic device may transfer card information, which has been stored in the same manner as an existing IC card, to the outside. More specifically, when the electronic device storing information of a payment card (for example, a credit card and a transportation card) approaches a payment machine, NFC-enabled mobile payment may be performed. When the electronic device storing information of an access card approaches an access machine, an access approval procedure may start. The credit card, the transportation card, the access card, etc. may be mounted on the security module in the applet, and the security module may store information of the cards mounted thereon. The information of the payment card may include at least one of a card number, balance, and details of usage. The information of the access card may include at least one of a user name, a user ID number, and an access history.

When the NFC module operates in the reader mode, the electronic device may read data from an external tag. In this instance, data the electronic device receives from the tag may be coded into a data exchange format defined in the NFC forum. Further, the NFC forum defines four record types. More specifically, the NFC forum defines four record type definitions (RTDs) including smart poster, text, uniform resource identifier (URI), and general control. When the data received from the tag is the smart poster type, the controller 180 may execute browser (for example, internet browser). When the data received from the tag is the text type, the controller 180 may execute a text viewer. When the data received from the tag is the URI type, the controller 180 may execute browser or make a call. When the data received from the tag is the general control type, the controller 180 may perform a proper operation depending on control contents.

When the NFC module operates in the P2P mode, the electronic device may perform P2P communication with other electronic device. In this instance, logical link control protocol (LLCP) may be applied to the P2P communication. The connection may be produced between the electronic device and other electronic device for the P2P communication. The connection may be divided into a connectionless mode, in which one packet switching is performed and ended, and a connection-oriented mode, in which packet switching is successively performed. Through the P2P communication, data, for example, electronic business cards, contact information, digital photographs, and URL, Bluetooth, a setup parameter for WiFi, etc. may be exchanged through the P2P communication. Because an available distance of the NFC communication is short, the P2P mode may be efficiently used to exchange data of small size.

Hereinafter, embodiments related to a control method which can be implemented in the electronic device configured as above are described with reference to the accompanying drawings. It is apparent to those skilled in the art that various modifications can be made to the disclosure without departing from the spirit and essential features of the present disclosure.

FIG. 6 illustrates another example of an electronic device related to an embodiment of the disclosure.

An electronic device 100 may include a main body 200 and a band portion 300 detachably coupled to the main body 200.

The main body 200 may include a display unit 151. Namely, the main body 200 may be a portion that includes a main substrate including various electronic equipments embedded therein, performs communication, processes information, and performs a function displayed on the display unit 151.

The main body 200 may include various input means. For example, at least one button 410 may be provided on a front surface or a side surface of the main body 200. The display unit 151 positioned on the front surface of the main body 200 may receive a touch operation of a user.

The band portion 300 may be a portion that is coupled with the main body 200 and is coupled with a user's wrist, etc. The main body 200 and the band portion 300 may have a shape corresponding to a shape of the user's wrist. The main body 200 and the band portion 300 may be made of a rigid material or a flexible material. For example, when the main body 200 and the band portion 300 are made of the rigid material, the band portion 300 may be formed in a curved shape so that it can be wound on the user's wrist, etc. In this instance, a connection portion 250 made of a flexible material may be provided on at least a portion of the band portion 300. When both the main body 200 and the band portion 300 are made of the rigid material, the connection portion 250 can secure a space in which the user's wrist, etc. can be put in or out when the main body 200 and the band portion 300 are coupled or separated. As another example, when the main body 200 and the band portion 300 are made of the flexible material, the band portion 300 may be configured in a shape corresponding to the user's wrist shape and/or a naturally curved material. For example, a synthetic resin, metal, a natural/artificial leather material, a material with high elasticity, or a combination thereof may be used.

The display unit 151 may be provided on the front surface of the main body 200. The display unit 151 can output information provided by a controller 180. The display unit 151 may include a touch sensor and may be implemented as a touch screen.

A printed circuit board (PCB) 185 and a battery 193 may be disposed on the band portion 300. The PCB 185 may be connected to the display unit 151 through a tape carrier package (TCP), and the like.

One side of the main body 200 and one side of the band portion 300 may be connected to one of a first coupling portion 400 and a second coupling portion 500. For example, as shown in FIG. 6, one side of the main body 200 may be connected to the first coupling portion 400, and one side of the band portion 300 may be connected to the second coupling portion 500. One end of the first coupling portion 400 and one end of the second coupling portion 500 may be configured to correspond to each other. The first coupling portion 400 and the second coupling portion 500 can be respectively connected to one side of the main body 200 and one side of the band portion 300 and can allow the main body 200 and the band portion 300 to be detachable.

Each of the first and second coupling portions 400 and 500 may be extended from one side of the main body 200 or one side of the band portion 300 and may form one body with the main body 200 or the band portion 300. Further, a space for accommodating the first and second coupling portions 400 and 500 may be provided on one side of the main body 200 or the band portion 300, and the first and second coupling portions 400 and 500 may be formed in a manner of being inserted in the space. The other side of the main body 200 and the other side of the band portion 300 that are connected to the first and second coupling portions 400 and 500 may be connected to each other and may form one body.

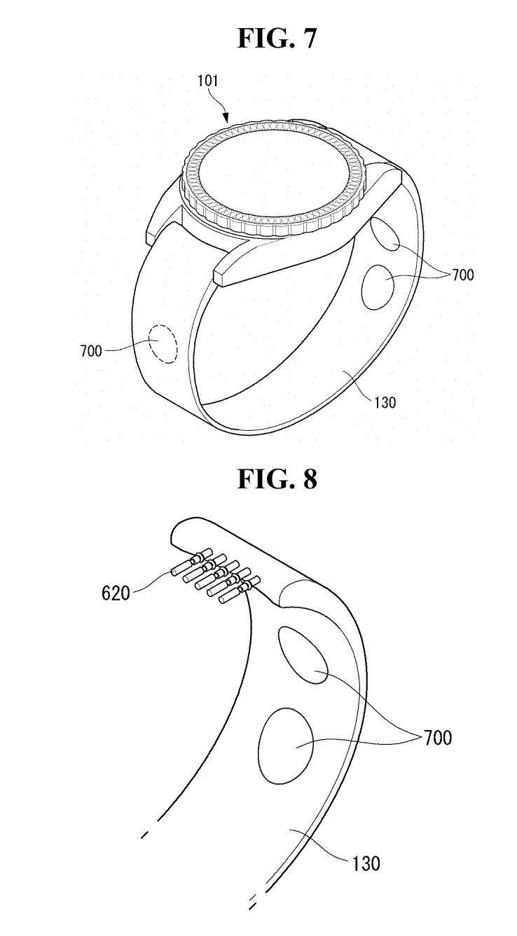

FIG. 7 illustrates an example of an electronic device according to an embodiment of the disclosure. More specifically, FIG. 7 illustrates a main body 101, a band portion 130, and electrodes 700.

The electrodes 700 may be provided on the band portion 130. The electrodes 700 may be positioned on a rear surface of the band portion 130. When the user wears the main body 101 and the band portion 130 on his/her wrist, the electrodes 700 may contact his/her wrist. The electrodes 700 may be configured to sense various biometric signals of the user. For example, the electrodes 700 may Electrodes 700 may be configured to measure electrocardiogram (ECG), galvanic skin response (GSR), and a skin temperature.

FIG. 8 illustrates an example of a band portion according to an embodiment of the disclosure. More specifically, FIG. 8 illustrates the band portion 130, the electrodes 700, and connector pins 620.

The electrodes 700 may be electrically connected to electric elements mounted inside the main body 101 through the connector pins 620. Namely, the electrodes 700 may be provided on the rear surface of the band portion 130 and may be electrically connected to the connector pins 620 through a flexible printed circuit board (FPCB) or electric wires mounted inside the band portion 130. At least one of the connector pins 620 may be electrically connected to the electrodes 700, and other connector pins 620 may be configured for the electrical connection between other electric elements of the main body 101 and the band portion 130.

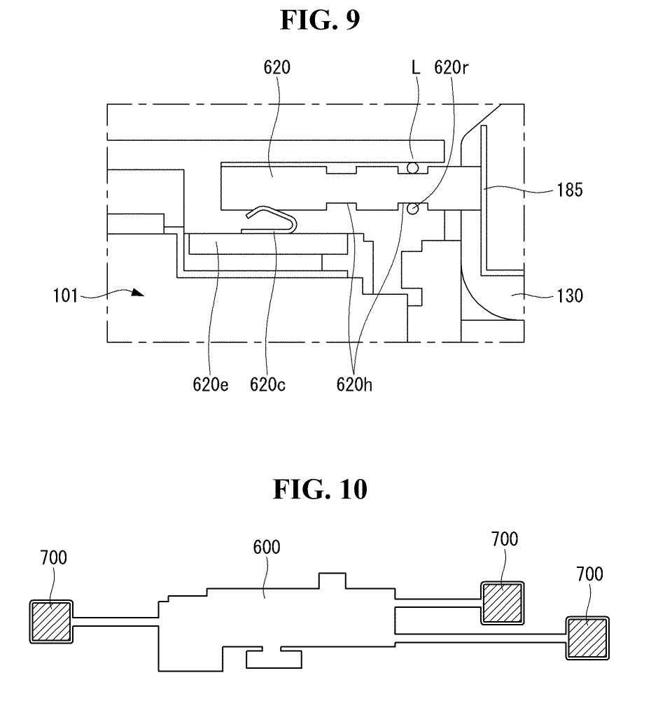

FIG. 9 illustrates an example of a cross section of an electronic device according to an embodiment of the disclosure. More specifically, FIG. 9 illustrates the connector pin 620, a FPCB 185, a pin groove 620h, a pin ring 620r, a pin clip 620c, and a PCB 620e.

The connector pin 620 electrically connected to the FPCB 185 of the band portion 130 may be inserted into the main body 101. One side of the connector pin 620 inserted into the main body 101 may be electrically connected to the PCB 620e provided on the main body 101 through the pin clip 620c. The connector pin 620 may have the pin groove 620h formed on an outer surface of the connector pin 620. The plurality of pin grooves 620h may be provided. The pin ring 620r may be inserted into at least one of the plurality of pin grooves 620h. When the connector pin 620 is inserted into the main body 101, the pin groove 620h and the pin ring 620r may receive a predetermined force or a predetermined pressure by a rib L provided inside the main body 101. Hence, inserting the connector pin 620 into the main body 101 can be maintained until a predetermined external force is applied to the connector pin 620.

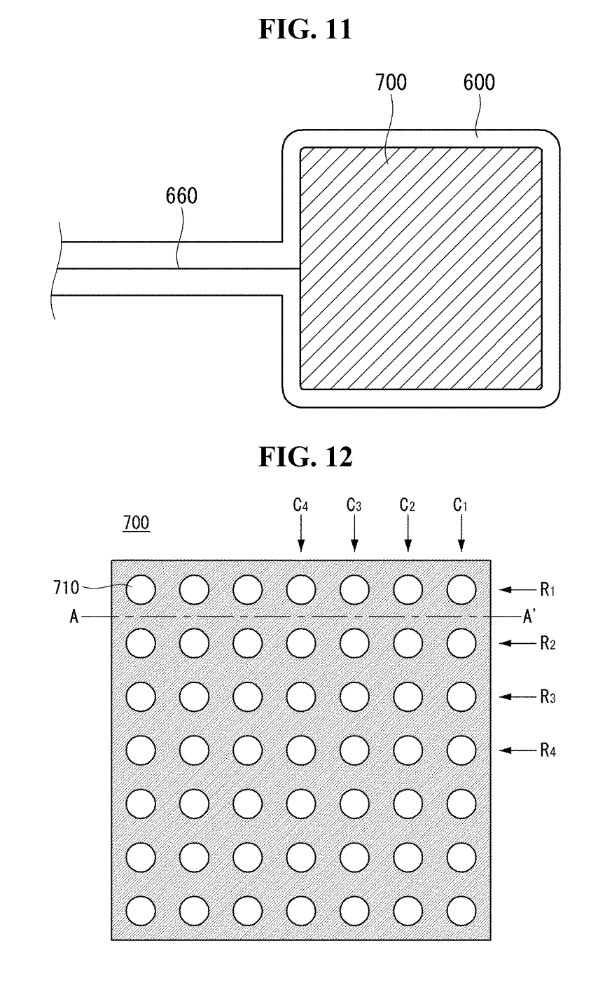

FIG. 10 illustrates an example of a FPCB and electrodes according to an embodiment of the disclosure. More specifically, FIG. 10 illustrates a FPCB 600 and the electrodes 700. FIG. 11 illustrates an example of an electrode according to an embodiment of the disclosure. More specifically, FIG. 11 illustrates the FPCB 600, electric wires 660, and the electrode 700.

The FPCB 600 may be mounted inside the main body 101 or may be mounted inside the band portion 130. The electrical connection between the FPCB 600 and the electrodes 700 can be implemented through various embodiments described above. The electrodes 700 may be provided in different directions around the FPCB 600. For example, one electrode 700 may be provided on one side of the FPCB 600, and two electrodes 700 may be provided on the other side of the FPCB 600. The number, the direction, the position, etc. of the electrodes 700 can be variously changed according to a biometric signal of the user to be measured.

FIGS. 12 to 43 illustrate various examples of an electrode according to an embodiment of the disclosure. More specifically, FIGS. 12 to 43 each illustrate a plane of an electrode.

The electrode may have a current flow area and a non-current flow area. The current flow area indicates an area where a current can flow, and the non-current flow area indicates an area where no current can flow. The current flow area and the non-current flow area may be distinguished from each other by being physically divided into two areas. Further, the current flow area and the non-current flow area may be distinguished from each other by forming two areas of different materials. For example, the current flow area may be formed of a conductive material, and the non-current flow area may be formed of a non-conductive material. In this instance, the current flow area and the non-current flow area may not be physically divided.

Referring to FIG. 12, the current flow area and the non-current flow area can be distinguished from each other by a circular vacancy 710. For example, the circular vacancy 710 may be formed using a sputtering method. An area where the circular vacancy 710 is formed may be the non-current flow area, and an area where the circular vacancy 710 is not formed may be the current flow area. The plurality of circular vacancies 710 may be provided in the electrode 700. The plurality of circular vacancies 710 may be formed on a first row R1. Further, the plurality of circular vacancies 710 may be formed on a first column C1. The electrode 700 may have a plurality of rows R. Further, the electrode 700 may have a plurality of columns C. The non-current flow area may be formed at intersections of the plurality of rows R and the plurality of columns C. Namely, the circular vacancies 710 may be formed at the intersections of the plurality of rows R and the plurality of columns C. For example, one circular vacancy 710 may be formed at an intersection of the first row R1 and the first column C1; one circular vacancy 710 may be formed at an intersection of the first row R1 and a second column C2; and one circular vacancy 710 may be formed at an intersection of the first row R1 and a third column C3. Thus, the plurality of circular vacancies 710 may form a pattern as a whole. The size of the circular vacancy 710 is not limited as long as the current flow area and the non-current flow area can be distinguished.

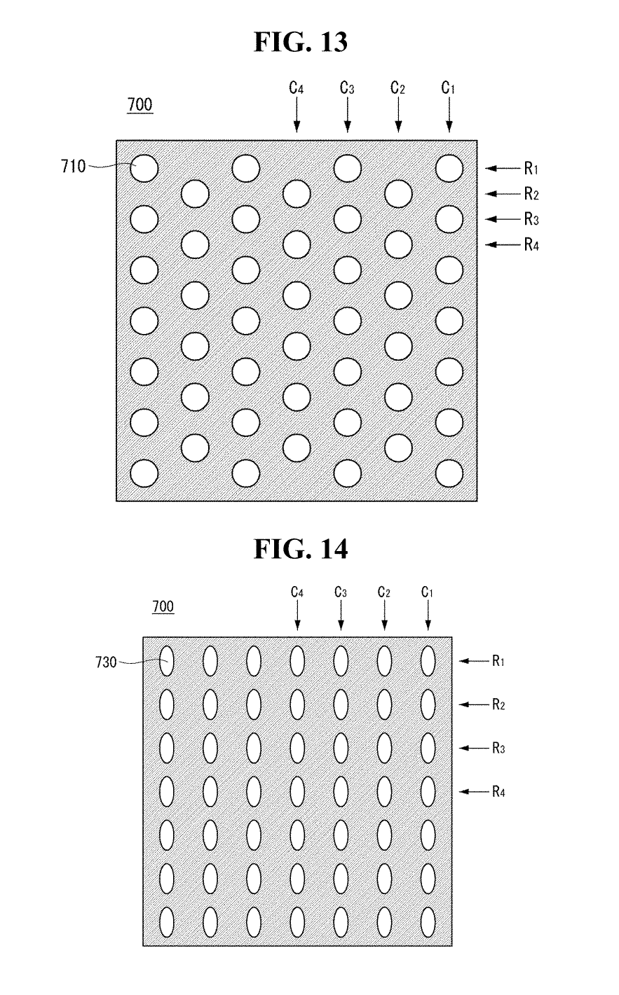

Referring to FIG. 13, the current flow area and the non-current flow area can be distinguished from each other by a circular vacancy 710. For example, the circular vacancy 710 may be formed using a sputtering method. An area where the circular vacancy 710 is formed may be the non-current flow area, and an area where the circular vacancy 710 is not formed may be the current flow area. The plurality of circular vacancies 710 may be provided in the electrode 700. The plurality of circular vacancies 710 may be formed on a first row R1. Further, the plurality of circular vacancies 710 may be formed on a first column C1. The electrode 700 may have a plurality of rows R. Further, the electrode 700 may have a plurality of columns C. The non-current flow area may be formed at intersections of the plurality of rows R and the plurality of columns C. Namely, the circular vacancies 710 may be formed at the intersections of the plurality of rows R and the plurality of columns C. In this instance, the circular vacancies 710 may be formed at intersections of odd-numbered rows R1 and R3 of the plurality of rows R and odd-numbered columns C1 and C3 of the plurality of columns C. Further, the circular vacancies 710 may be formed at intersections of even-numbered rows R2 and R4 of the plurality of rows R and even-numbered columns C2 and C4 of the plurality of columns C. For example, one circular vacancy 710 may be formed at an intersection of the first row R1 and the first column C1; the circular vacancy 710 may not be formed at an intersection of the first row R1 and the second column C2; and one circular vacancy 710 may be formed at an intersection of the first row R1 and the third column C3. Thus, the plurality of circular vacancies 710 may form a pattern as a whole. The size of the circular vacancy 710 is not limited as long as the current flow area and the non-current flow area can be distinguished.

Referring to FIG. 14, the current flow area and the non-current flow area can be distinguished from each other by an elliptical vacancy 730. For example, the elliptical vacancy 730 may be formed using a sputtering method. An area where the elliptical vacancy 730 is formed may be the non-current flow area, and an area where the elliptical vacancy 730 is not formed may be the current flow area. The plurality of elliptical vacancies 730 may be provided in the electrode 700. The plurality of elliptical vacancies 730 may be formed on a first row R1. Further, the plurality of elliptical vacancies 730 may be formed on a first column C1. The electrode 700 may have a plurality of rows R. Further, the electrode 700 may have a plurality of columns C. The non-current flow area may be formed at intersections of the plurality of rows R and the plurality of columns C. Namely, the elliptical vacancies 730 may be formed at the intersections of the plurality of rows R and the plurality of columns C. For example, one elliptical vacancy 730 may be formed at an intersection of the first row R1 and the first column C1; one elliptical vacancy 730 may be formed at an intersection of the first row R1 and a second column C2; and one elliptical vacancy 730 may be formed at an intersection of the first row R1 and a third column C3. Thus, the plurality of elliptical vacancies 730 may form a pattern as a whole. The size of the elliptical vacancy 730 is not limited as long as the current flow area and the non-current flow area can be distinguished.

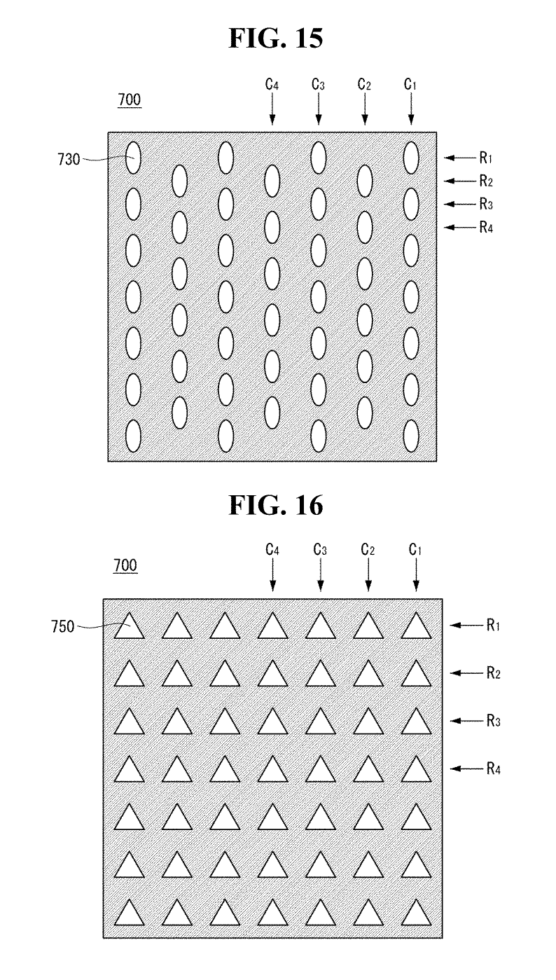

Referring to FIG. 15, the current flow area and the non-current flow area can be distinguished from each other by an elliptical vacancy 730. For example, the elliptical vacancy 730 may be formed using a sputtering method. An area where the elliptical vacancy 730 is formed may be the non-current flow area, and an area where the elliptical vacancy 730 is not formed may be the current flow area. The plurality of elliptical vacancies 730 may be provided in the electrode 700. The plurality of elliptical vacancies 730 may be formed on a first row R1. Further, the plurality of elliptical vacancies 730 may be formed on a first column C1. The electrode 700 may have a plurality of rows R. Further, the electrode 700 may have a plurality of columns C. The non-current flow area may be formed at intersections of the plurality of rows R and the plurality of columns C. Namely, the elliptical vacancies 730 may be formed at the intersections of the plurality of rows R and the plurality of columns C. In this instance, the elliptical vacancies 730 may be formed at intersections of odd-numbered rows R1 and R3 of the plurality of rows R and odd-numbered columns C1 and C3 of the plurality of columns C. Further, the elliptical vacancies 730 may be formed at intersections of even-numbered rows R2 and R4 of the plurality of rows R and even-numbered columns C2 and C4 of the plurality of columns C. For example, one elliptical vacancy 730 may be formed at an intersection of the first row R1 and the first column C1; the elliptical vacancy 730 may not be formed at an intersection of the first row R1 and the second column C2; and one elliptical vacancy 730 may be formed at an intersection of the first row R1 and the third column C3. Thus, the plurality of elliptical vacancies 730 may form a pattern as a whole. The size of the elliptical vacancy 730 is not limited as long as the current flow area and the non-current flow area can be distinguished.

Referring to FIG. 16, the current flow area and the non-current flow area can be distinguished from each other by a triangular vacancy 750. For example, the triangular vacancy 750 may be formed using a sputtering method. An area where the triangular vacancy 750 is formed may be the non-current flow area, and an area where the triangular vacancy 750 is not formed may be the current flow area. The plurality of triangular vacancies 750 may be provided in the electrode 700. The plurality of triangular vacancies 750 may be formed on a first row R1. Further, the plurality of triangular vacancies 750 may be formed on a first column C1. The electrode 700 may have a plurality of rows R. Further, the electrode 700 may have a plurality of columns C. The non-current flow area may be formed at intersections of the plurality of rows R and the plurality of columns C. Namely, the triangular vacancies 750 may be formed at the intersections of the plurality of rows R and the plurality of columns C. For example, one triangular vacancy 750 may be formed at an intersection of the first row R1 and the first column C1; one triangular vacancy 750 may be formed at an intersection of the first row R1 and a second column C2; and one triangular vacancy 750 may be formed at an intersection of the first row R1 and a third column C3. Thus, the plurality of triangular vacancies 750 may form a pattern as a whole. The size of the triangular vacancy 750 is not limited as long as the current flow area and the non-current flow area can be distinguished.

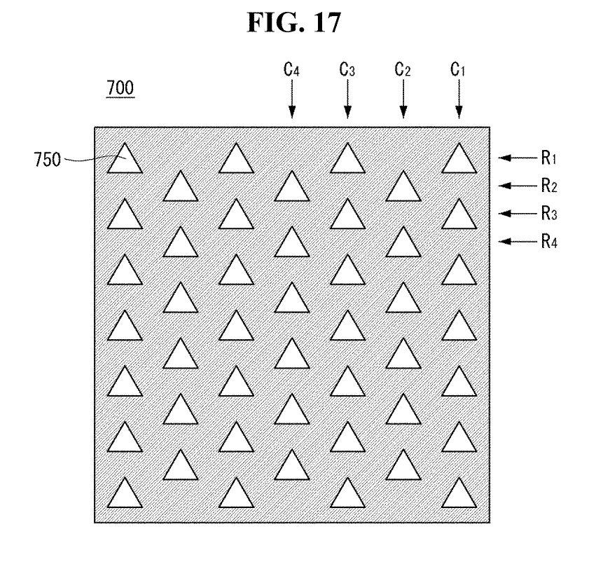

Referring to FIG. 17, the current flow area and the non-current flow area can be distinguished from each other by a triangular vacancy 750. For example, the triangular vacancy 750 may be formed using a sputtering method. An area where the triangular vacancy 750 is formed may be the non-current flow area, and an area where the triangular vacancy 750 is not formed may be the current flow area. The plurality of triangular vacancies 750 may be provided in the electrode 700. The plurality of triangular vacancies 750 may be formed on a first row R1. Further, the plurality of triangular vacancies 750 may be formed on a first column C1. The electrode 700 may have a plurality of rows R. Further, the electrode 700 may have a plurality of columns C. The non-current flow area may be formed at intersections of the plurality of rows R and the plurality of columns C. Namely, the triangular vacancies 750 may be formed at the intersections of the plurality of rows R and the plurality of columns C. In this instance, the triangular vacancies 750 may be formed at intersections of odd-numbered rows R1 and R3 of the plurality of rows R and odd-numbered columns C1 and C3 of the plurality of columns C. Further, the triangular vacancies 750 may be formed at intersections of even-numbered rows R2 and R4 of the plurality of rows R and even-numbered columns C2 and C4 of the plurality of columns C. For example, one triangular vacancy 750 may be formed at an intersection of the first row R1 and the first column C1; the triangular vacancy 750 may not be formed at an intersection of the first row R1 and the second column C2; and one triangular vacancy 750 may be formed at an intersection of the first row R1 and the third column C3. Thus, the plurality of triangular vacancies 750 may form a pattern as a whole. The size of the triangular vacancy 750 is not limited as long as the current flow area and the non-current flow area can be distinguished.

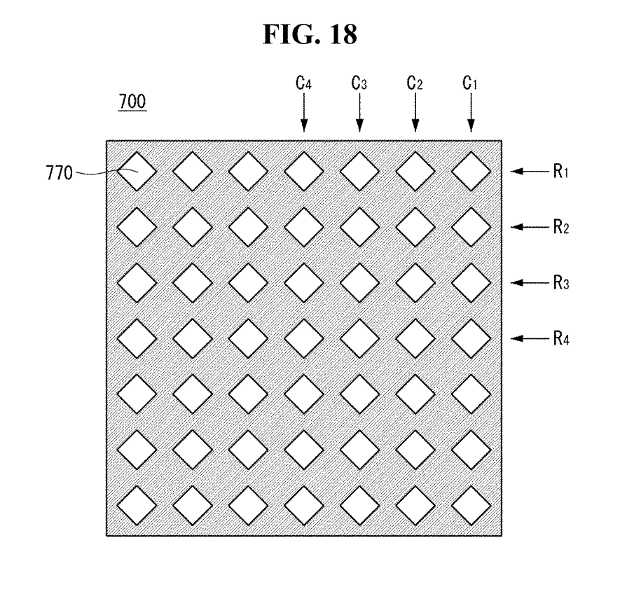

Referring to FIG. 18, the current flow area and the non-current flow area can be distinguished from each other by a rhombus vacancy 770. For example, the rhombus vacancy 770 may be formed using a sputtering method. An area where the rhombus vacancy 770 is formed may be the non-current flow area, and an area where the rhombus vacancy 770 is not formed may be the current flow area. The plurality of rhombus vacancies 770 may be provided in the electrode 700. The plurality of rhombus vacancies 770 may be formed on a first row R1. Further, the plurality of rhombus vacancies 770 may be formed on a first column C1. The electrode 700 may have a plurality of rows R. Further, the electrode 700 may have a plurality of columns C. The non-current flow area may be formed at intersections of the plurality of rows R and the plurality of columns C. Namely, the rhombus vacancies 770 may be formed at the intersections of the plurality of rows R and the plurality of columns C. For example, one rhombus vacancy 770 may be formed at an intersection of the first row R1 and the first column C1; one rhombus vacancy 770 may be formed at an intersection of the first row R1 and a second column C2; and one rhombus vacancy 770 may be formed at an intersection of the first row R1 and a third column C3. Thus, the plurality of rhombus vacancies 770 may form a pattern as a whole. The size of the rhombus vacancy 770 is not limited as long as the current flow area and the non-current flow area can be distinguished.

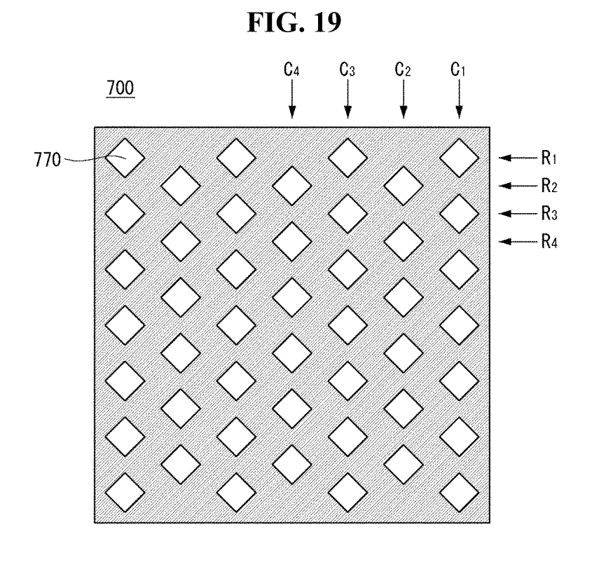

Referring to FIG. 19, the current flow area and the non-current flow area can be distinguished from each other by a rhombus vacancy 770. For example, the rhombus vacancy 770 may be formed using a sputtering method. An area where the rhombus vacancy 770 is formed may be the non-current flow area, and an area where the rhombus vacancy 770 is not formed may be the current flow area. The plurality of rhombus vacancies 770 may be provided in the electrode 700. The plurality of rhombus vacancies 770 may be formed on a first row R1. Further, the plurality of rhombus vacancies 770 may be formed on a first column C1. The electrode 700 may have a plurality of rows R. Further, the electrode 700 may have a plurality of columns C. The non-current flow area may be formed at intersections of the plurality of rows R and the plurality of columns C. Namely, the rhombus vacancies 770 may be formed at the intersections of the plurality of rows R and the plurality of columns C. In this instance, the rhombus vacancies 770 may be formed at intersections of odd-numbered rows R1 and R3 of the plurality of rows R and odd-numbered columns C1 and C3 of the plurality of columns C. Further, the rhombus vacancies 770 may be formed at intersections of even-numbered rows R2 and R4 of the plurality of rows R and even-numbered columns C2 and C4 of the plurality of columns C. For example, one rhombus vacancy 770 may be formed at an intersection of the first row R1 and the first column C1; the rhombus vacancy 770 may not be formed at an intersection of the first row R1 and the second column C2; and one rhombus vacancy 770 may be formed at an intersection of the first row R1 and the third column C3. Thus, the plurality of rhombus vacancies 770 may form a pattern as a whole. The size of the rhombus vacancy 770 is not limited as long as the current flow area and the non-current flow area can be distinguished.

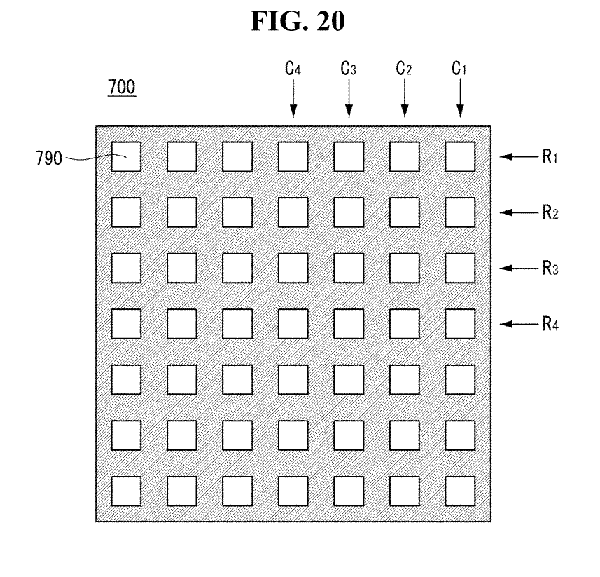

Referring to FIG. 20, the current flow area and the non-current flow area can be distinguished from each other by a rectangular vacancy 790. For example, the rectangular vacancy 790 may be formed using a sputtering method. An area where the rectangular vacancy 790 is formed may be the non-current flow area, and an area where the rectangular vacancy 790 is not formed may be the current flow area. The plurality of rectangular vacancies 790 may be provided in the electrode 700. The plurality of rectangular vacancies 790 may be formed on a first row R1. Further, the plurality of rectangular vacancies 790 may be formed on a first column C1. The electrode 700 may have a plurality of rows R. Further, the electrode 700 may have a plurality of columns C. The non-current flow area may be formed at intersections of the plurality of rows R and the plurality of columns C. Namely, the rectangular vacancies 790 may be formed at the intersections of the plurality of rows R and the plurality of columns C. For example, one rectangular vacancy 790 may be formed at an intersection of the first row R1 and the first column C1; one rectangular vacancy 790 may be formed at an intersection of the first row R1 and a second column C2; and one rectangular vacancy 790 may be formed at an intersection of the first row R1 and a third column C3. Thus, the plurality of rectangular vacancies 790 may form a pattern as a whole. The size of the rectangular vacancy 790 is not limited as long as the current flow area and the non-current flow area can be distinguished.

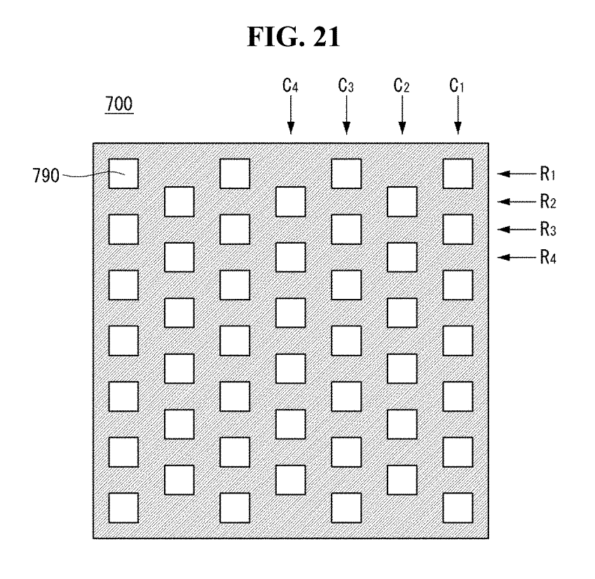

Referring to FIG. 21, the current flow area and the non-current flow area can be distinguished from each other by a rectangular vacancy 790. For example, the rectangular vacancy 790 may be formed using a sputtering method. An area where the rectangular vacancy 790 is formed may be the non-current flow area, and an area where the rectangular vacancy 790 is not formed may be the current flow area. The plurality of rectangular vacancies 790 may be provided in the electrode 700. The plurality of rectangular vacancies 790 may be formed on a first row R1. Further, the plurality of rectangular vacancies 790 may be formed on a first column C1. The electrode 700 may have a plurality of rows R. Further, the electrode 700 may have a plurality of columns C. The non-current flow area may be formed at intersections of the plurality of rows R and the plurality of columns C. Namely, the rectangular vacancies 790 may be formed at the intersections of the plurality of rows R and the plurality of columns C. In this instance, the rectangular vacancies 790 may be formed at intersections of odd-numbered rows R1 and R3 of the plurality of rows R and odd-numbered columns C1 and C3 of the plurality of columns C. Further, the rectangular vacancies 790 may be formed at intersections of even-numbered rows R2 and R4 of the plurality of rows R and even-numbered columns C2 and C4 of the plurality of columns C. For example, one rectangular vacancy 790 may be formed at an intersection of the first row R1 and the first column C1; the rectangular vacancy 790 may not be formed at an intersection of the first row R1 and the second column C2; and one rectangular vacancy 790 may be formed at an intersection of the first row R1 and the third column C3. Thus, the plurality of rectangular vacancies 790 may form a pattern as a whole. The size of the rectangular vacancy 790 is not limited as long as the current flow area and the non-current flow area can be distinguished.

Hereinafter, the description duplicated with that described above is omitted, and only a difference will be described.

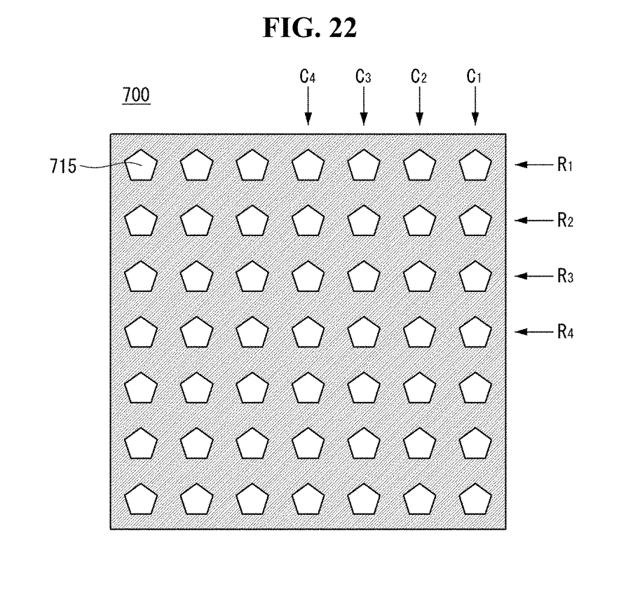

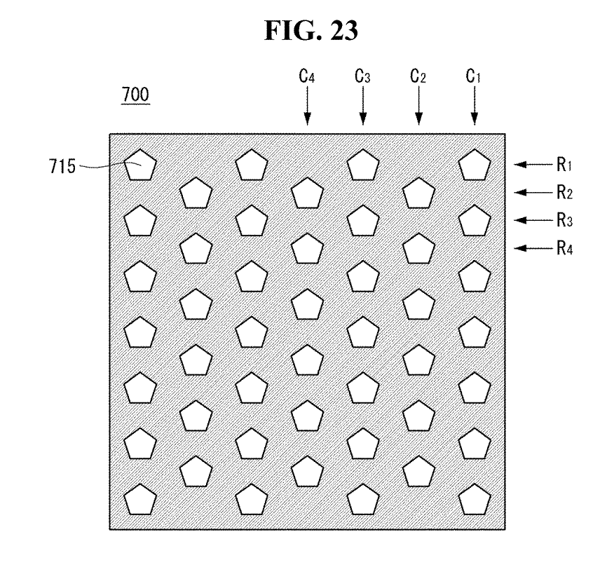

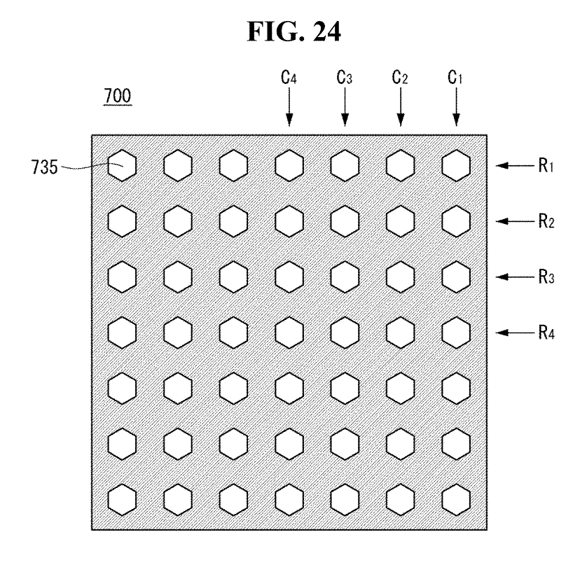

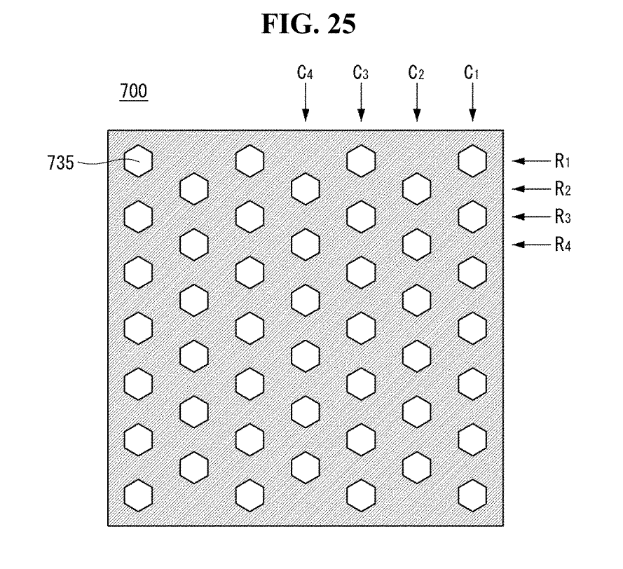

Referring to FIGS. 22 and 23, the current flow area and the non-current flow area can be distinguished from each other by a pentagonal vacancy 715. Referring to FIGS. 24 and 25, the current flow area and the non-current flow area can be distinguished from each other by a hexagonal vacancy 735.

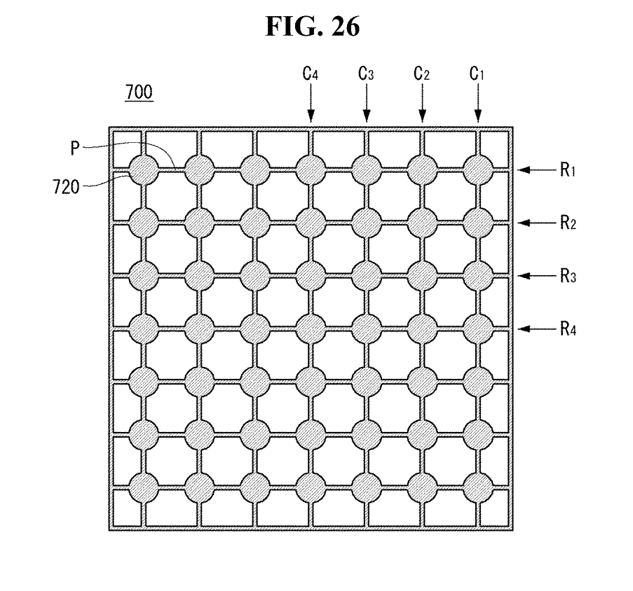

Referring to FIG. 26, the current flow area and the non-current flow area can be distinguished from each other by a circular plate 720. For example, the circular plate 720 may be formed using a sputtering method. An area where the circular plate 720 is formed may be the current flow area, and an area where the circular plate 720 is not formed may be the non-current flow area. The plurality of circular plates 720 may be provided in the electrode 700. The plurality of circular plates 720 may be formed on a first row R1. Further, the plurality of circular plates 720 may be formed on a first column C1. The electrode 700 may have a plurality of rows R. Further, the electrode 700 may have a plurality of columns C. The current flow area may be formed at intersections of the plurality of rows R and the plurality of columns C. Namely, the circular plates 720 may be formed at the intersections of the plurality of rows R and the plurality of columns C. For example, one circular plate 720 may be formed at an intersection of the first row R1 and the first column C1; one circular plate 720 may be formed at an intersection of the first row R1 and a second column C2; and one circular plate 720 may be formed at an intersection of the first row R1 and a third column C3. Thus, the plurality of circular plates 720 may form a pattern as a whole. The size of the circular plate 720 is not limited as long as the current flow area and the non-current flow area can be distinguished. The plurality of circular plates 720 may be electrically connected to one another to form the current flow area. For example, one circular plate 720 and another circular plate 720 may be connected by a current flow path P.

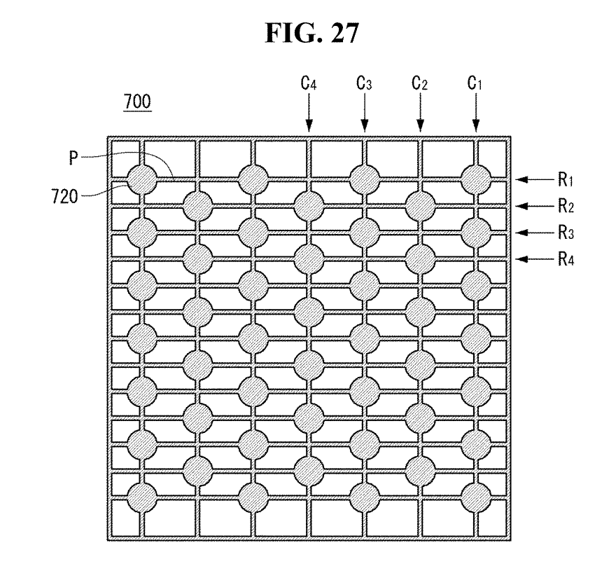

Referring to FIG. 27, the current flow area and the non-current flow area can be distinguished from each other by a circular plate 720. For example, the circular plate 720 may be formed using a sputtering method. An area where the circular plate 720 is formed may be the current flow area, and an area where the circular plate 720 is not formed may be the non-current flow area. The plurality of circular plates 720 may be provided in the electrode 700. The plurality of circular plates 720 may be formed on a first row R1. Further, the plurality of circular plates 720 may be formed on a first column C1. The electrode 700 may have a plurality of rows R. Further, the electrode 700 may have a plurality of columns C. The current flow area may be formed at intersections of the plurality of rows R and the plurality of columns C. Namely, the circular plates 720 may be formed at the intersections of the plurality of rows R and the plurality of columns C. In this instance, the circular plates 720 may be formed at intersections of odd-numbered rows R1 and R3 of the plurality of rows R and odd-numbered columns C1 and C3 of the plurality of columns C. Further, the circular plates 720 may be formed at intersections of even-numbered rows R2 and R4 of the plurality of rows R and even-numbered columns C2 and C4 of the plurality of columns C. For example, one circular plate 720 may be formed at an intersection of the first row R1 and the first column C1; the circular plate 720 may not be formed at an intersection of the first row R1 and the second column C2; and one circular plate 720 may be formed at an intersection of the first row R1 and the third column C3. Thus, the plurality of circular plates 720 may form a pattern as a whole. The size of the circular plate 720 is not limited as long as the current flow area and the non-current flow area can be distinguished. The plurality of circular plates 720 may be electrically connected to one another to form the current flow area. For example, one circular plate 720 and another circular plate 720 may be connected by a current flow path P.

Hereinafter, the description duplicated with that described above is omitted, and only a difference will be described.

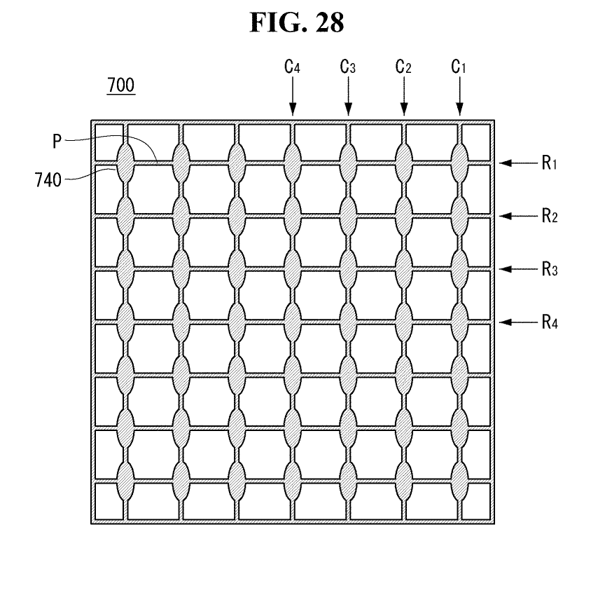

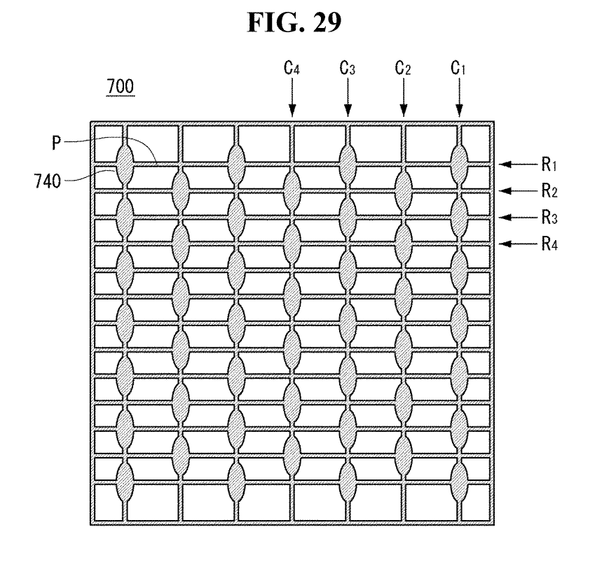

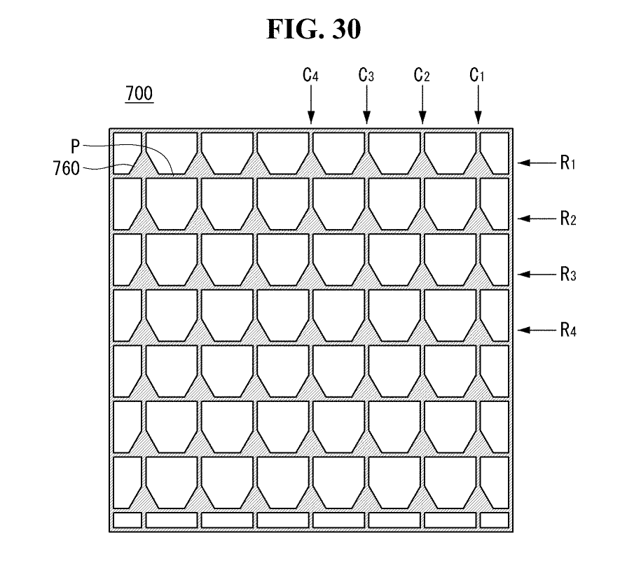

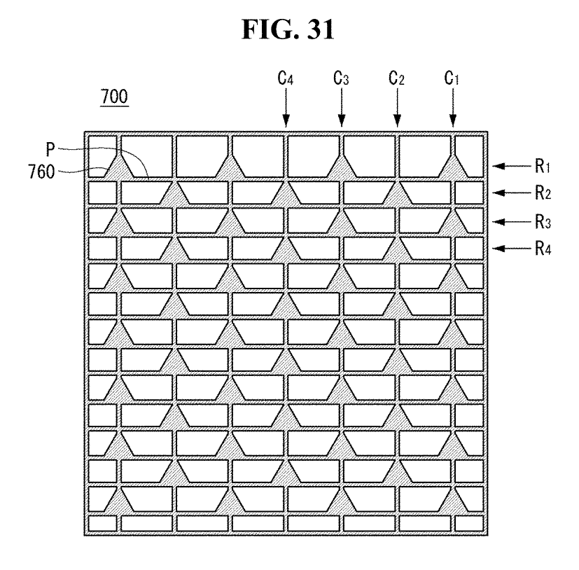

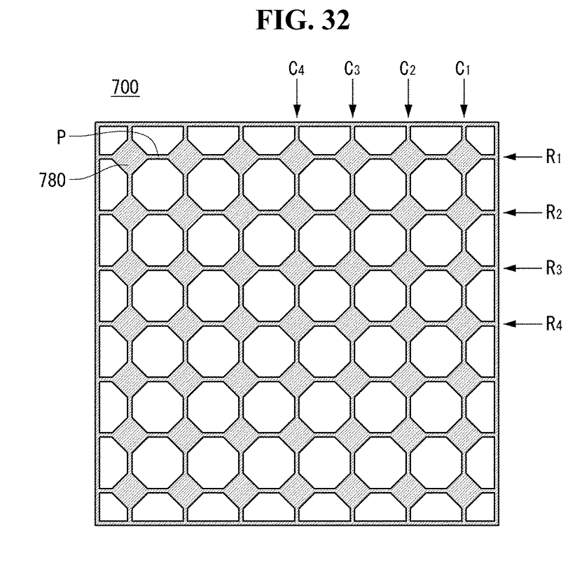

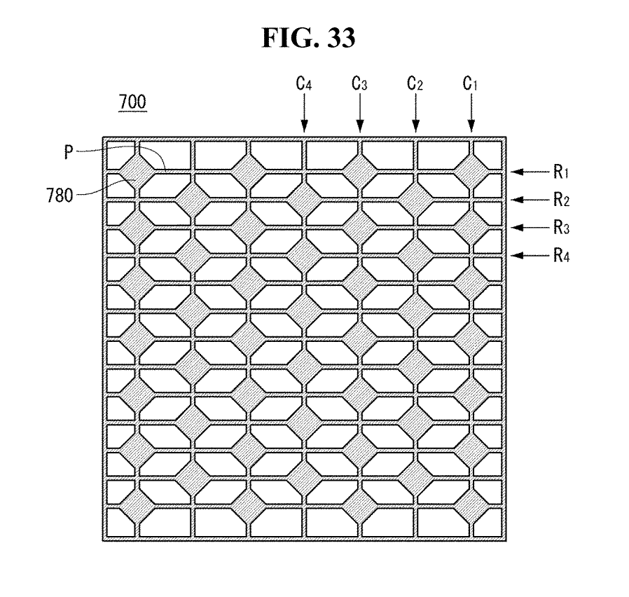

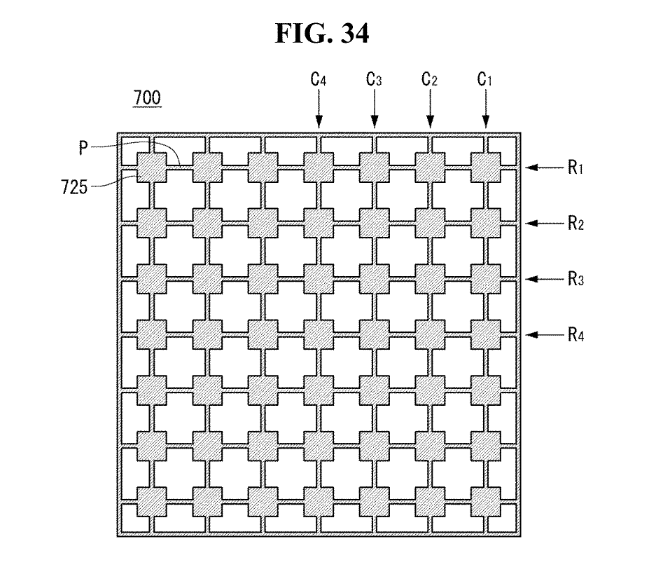

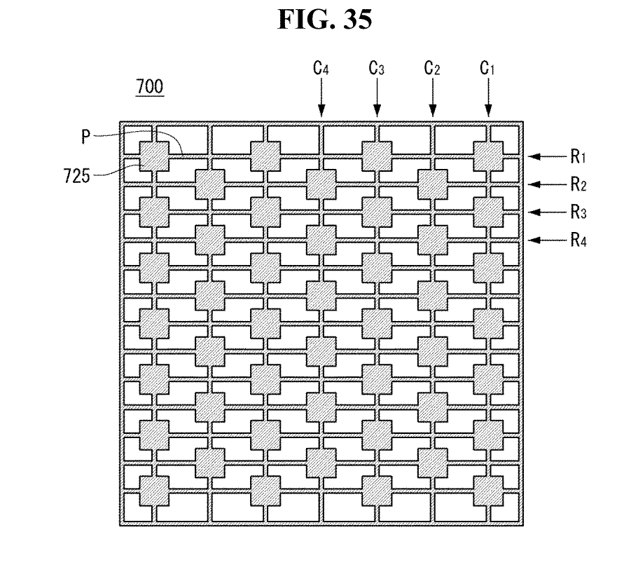

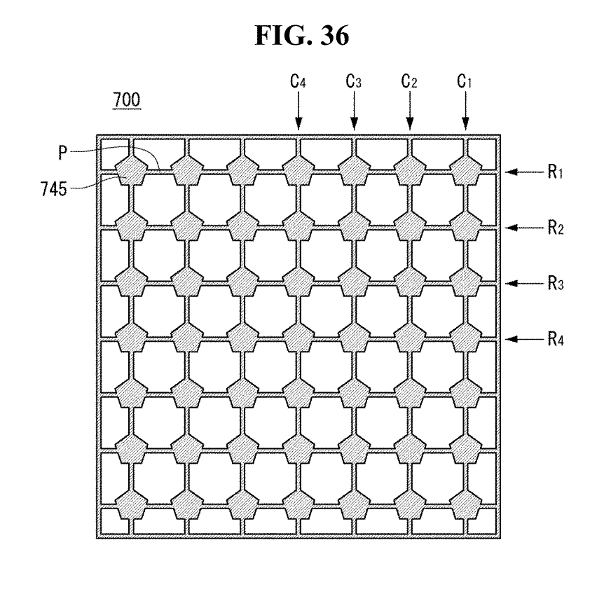

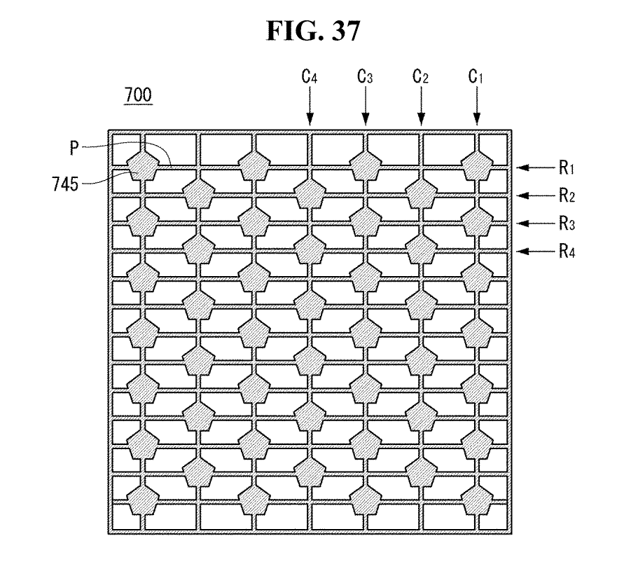

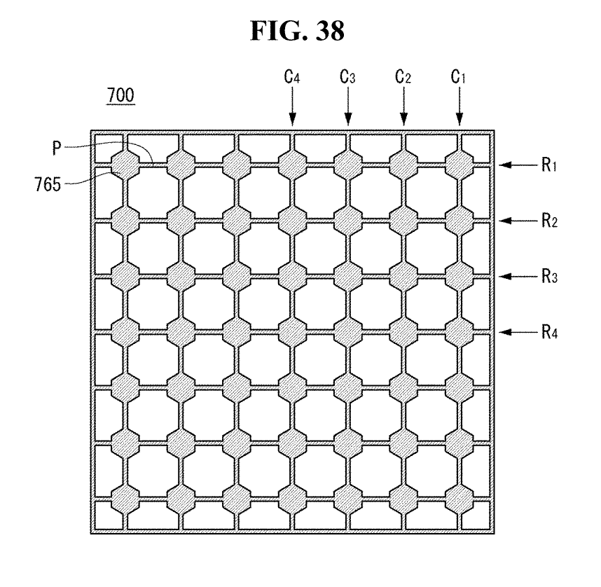

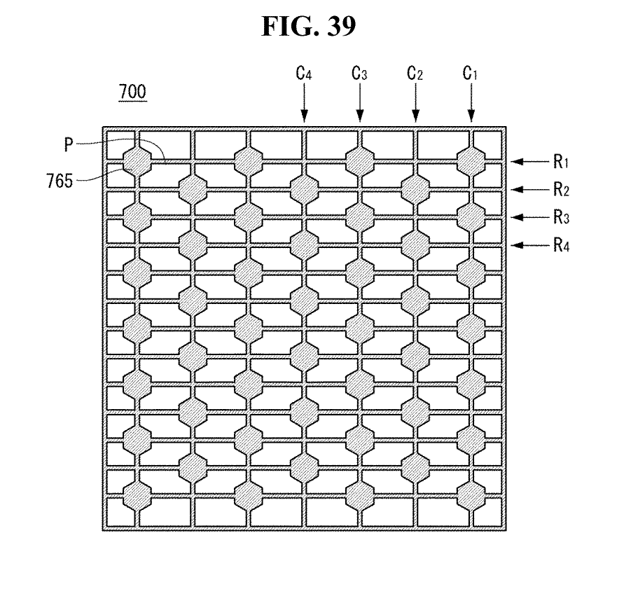

Referring to FIGS. 28 and 29, the current flow area and the non-current flow area can be distinguished from each other by an elliptical plate 740. Referring to FIGS. 30 and 31, the current flow area and the non-current flow area can be distinguished from each other by a triangular plate 760. Referring to FIGS. 32 and 33, the current flow area and the non-current flow area can be distinguished from each other by a rhombus plate 780. Referring to FIGS. 34 and 35, the current flow area and the non-current flow area can be distinguished from each other by a rectangular plate 725. Referring to FIGS. 36 and 37, the current flow area and the non-current flow area can be distinguished from each other by a pentagonal plate 745. Referring to FIGS. 38 and 39, the current flow area and the non-current flow area can be distinguished from each other by a hexagonal plate 765.

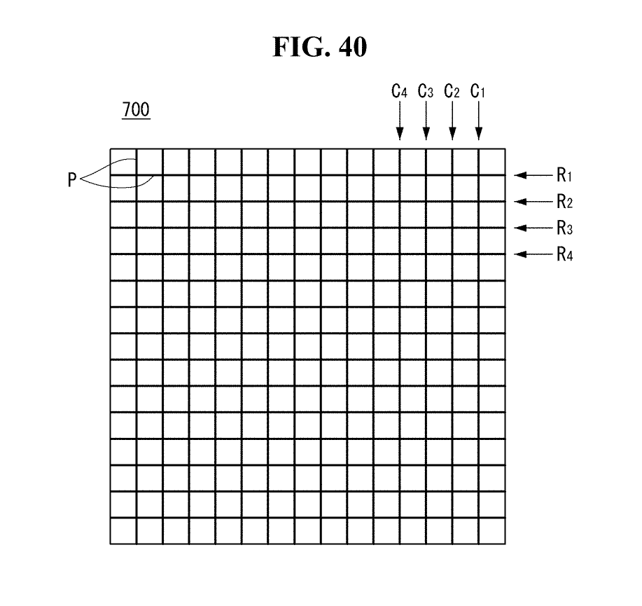

Referring to FIG. 40, the current flow area and the non-current flow area can be distinguished from each other by a current flow path P. For example, the current flow path P may be formed using a sputtering method. An area where the current flow path P is formed may be the current flow area, and an area where the current flow path P is not formed may be the non-current flow area. The plurality of current flow paths P may be provided in the electrode 700. One current flow path P may be formed on a first row R1. Further, one current flow path P may be formed on a first column C1. The electrode 700 may have a plurality of rows R. Further, the electrode 700 may have a plurality of columns C. The current flow area may be formed at intersections of the plurality of rows R and the plurality of columns C. Thus, the plurality of current flow paths P may form a pattern as a whole. A width of the current flow path P is not limited as long as the current flow area and the non-current flow area can be distinguished.

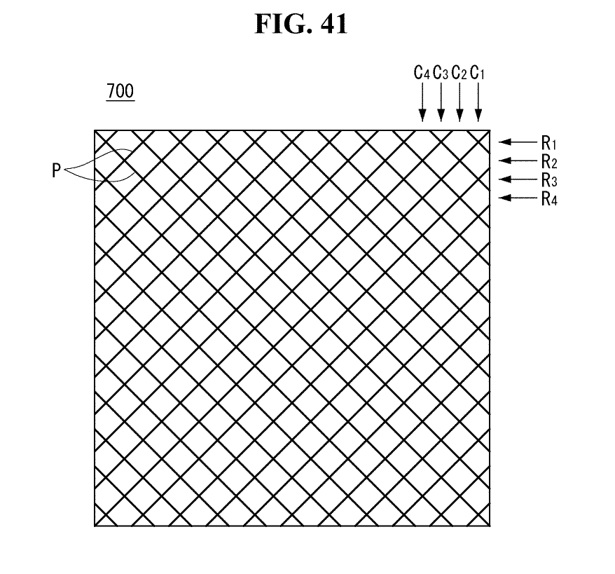

Referring to FIG. 41, the current flow area and the non-current flow area can be distinguished from each other by a current flow path P. For example, the current flow path P may be formed using a sputtering method. An area where the current flow path P is formed may be the current flow area, and an area where the current flow path P is not formed may be the non-current flow area. The plurality of current flow paths P may be provided in the electrode 700. A crossing path of the plurality of current flow paths P may be formed on a first row R1. Further, a crossing path of the plurality of current flow paths P may be formed on a first column C1. The electrode 700 may have a plurality of rows R. Further, the electrode 700 may have a plurality of columns C. Thus, the plurality of current flow paths P may form a pattern as a whole. A width of the current flow path P is not limited as long as the current flow area and the non-current flow area can be distinguished.

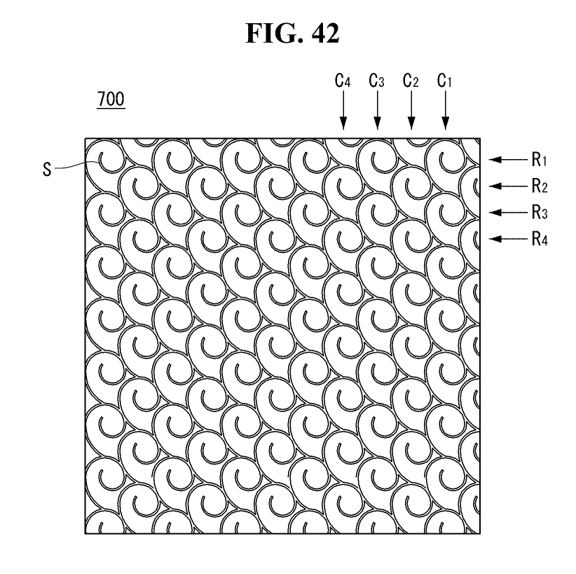

Referring to FIG. 42, the current flow area and the non-current flow area can be distinguished from each other by a spiral S. For example, the spiral S may be formed using a sputtering method. An area where the spiral S is formed may be the current flow area, and an area where the spiral S is not formed may be the non-current flow area. The plurality of spirals S may be provided in the electrode 700. The plurality of spirals S may be formed on a first row R1. Further, the plurality of spirals S may be formed on a first column C1. The electrode 700 may have a plurality of rows R. Further, the electrode 700 may have a plurality of columns C. The current flow area may be formed at intersections of the plurality of rows R and the plurality of columns C. Namely, the spirals S may be formed at the intersections of the plurality of rows R and the plurality of columns C. In this instance, the spirals S may be formed at intersections of odd-numbered rows R1 and R3 of the plurality of rows R and odd-numbered columns C1 and C3 of the plurality of columns C. Further, the spirals S may be formed at intersections of even-numbered rows R2 and R4 of the plurality of rows R and even-numbered columns C2 and C4 of the plurality of columns C. For example, one spiral S may be formed at an intersection of the first row R1 and the first column C1; the spiral S may not be formed at an intersection of the first row R1 and the second column C2; and one spiral S may be formed at an intersection of the first row R1 and the third column C3. Thus, the plurality of spirals S may form a pattern as a whole. A width of the spiral S is not limited as long as the current flow area and the non-current flow area can be distinguished. The plurality of spirals S may be electrically connected to one another to form the current flow area.

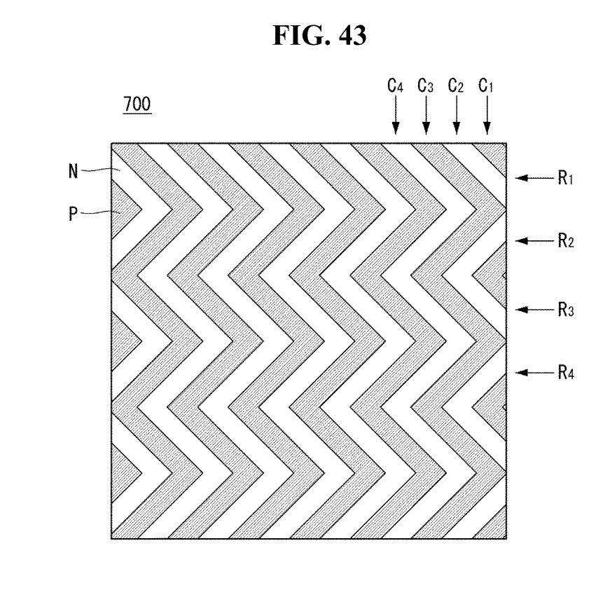

Referring to FIG. 43, the current flow area and the non-current flow area can be distinguished from each other by a zigzag P. For example, the zigzag P may be formed using a sputtering method. An area where the zigzag P is formed may be the current flow area, and an area N where the zigzag P is not formed may be the non-current flow area. The plurality of zigzags P may be provided in the electrode 700. The electrode 700 may have a plurality of rows R. Further, the electrode 700 may have a plurality of columns C.

At a first row R1, the current flow area on odd-numbered columns C of the plurality of columns C may be formed to be inclined from a left upper side to a right lower side. Further, at the first row R1, the non-current flow area on even-numbered columns C of the plurality of columns C may be formed to be inclined from the left upper side to the right lower side.

At a second row R2, the current flow area on the odd-numbered columns C of the plurality of columns C may be formed to be inclined from a right upper side to a left lower side. Further, at the second row R2, the non-current flow area on the even-numbered columns C of the plurality of columns C may be formed to be inclined from the right upper side to the left lower side.

At a third row R3, the current flow area on the odd-numbered columns C of the plurality of columns C may be formed to be inclined from the left upper side to the right lower side. Further, at the third row R3, the non-current flow area on the even-numbered columns C of the plurality of columns C may be formed to be inclined from the left upper side to the right lower side.

The above-described slopes may be formed steeply. The fact that the slopes are formed steeply may mean that edges of the zigzag are sharpened. Hence, a resistance of the electrode can increase.

Through the above-described configuration, the plurality of zigzags P may form a pattern as a whole. A width of the zigzags P is not limited as long as the current flow area and the non-current flow area can be distinguished.

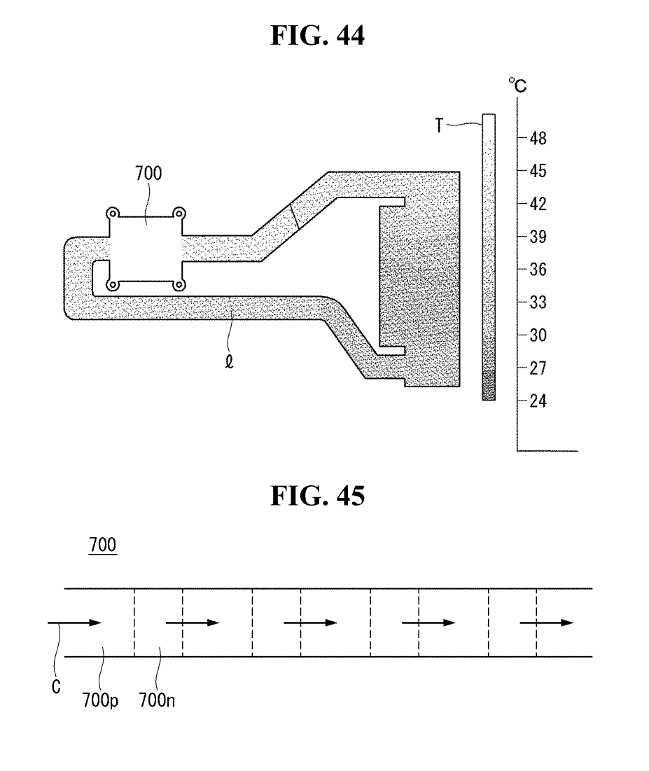

FIG. 44 illustrates an example of a temperature distribution of an electrode according to an embodiment of the disclosure. More specifically, FIG. 44 illustrates the electrode 700, a color temperature T, and the electric wire 1.

The color temperature T indicates a temperature distribution of the electrode 700 or the electric wire 1 using different colors. For example, the higher the temperature is, the lighter the color is, and the lower the temperature is, the darker the color is. When a voltage applied to the electrode 700 increases, a temperature of the electrode 700 may increase to about 45° C. or higher. On the other hand, the electric wire 1 may have a temperature lower than about 45° C. In other words, when the voltage is applied to the electrode 700 according to the embodiment of the disclosure, the temperature of the electrode 700 may be much higher than a temperature of another conductor, for example, the electric wire 1.

Alternatively, the electrode 700 may include a heating element. For example, the heating element included in the electrode 700 may be a heater. The controller 180 can achieve the above-described effect by increasing or decreasing a temperature of the heating element.

FIG. 45 illustrates an example of a cross section of an electrode according to an embodiment of the disclosure. More specifically, FIG. 45 illustrates an example of a cross-section of, for example, line A-A′ of FIG. 12. Further, FIG. 45 illustrates the electrode 700, a current C, a current flow area 700p, and a non-current flow area 700n.

When a current flows in the electrode 700 in which the non-current flow area 700n is formed, an electrical resistance of the electrode 700 having the non-current flow area 700n may be greater than an electrical resistance of the electrode 700 not having the non-current flow area 700n. In other words, when a current flows in the electrode 700 in which the current flow area 700p and the non-current flow area 700n are formed, an electrical resistance of the electrode 700 having the current flow area 700p and the non-current flow area 700n may be greater than an electrical resistance of the electrode 700 having only the current flow area 700p. Hence, the electrode 700 according to the embodiment of the disclosure can provide a high resistance.

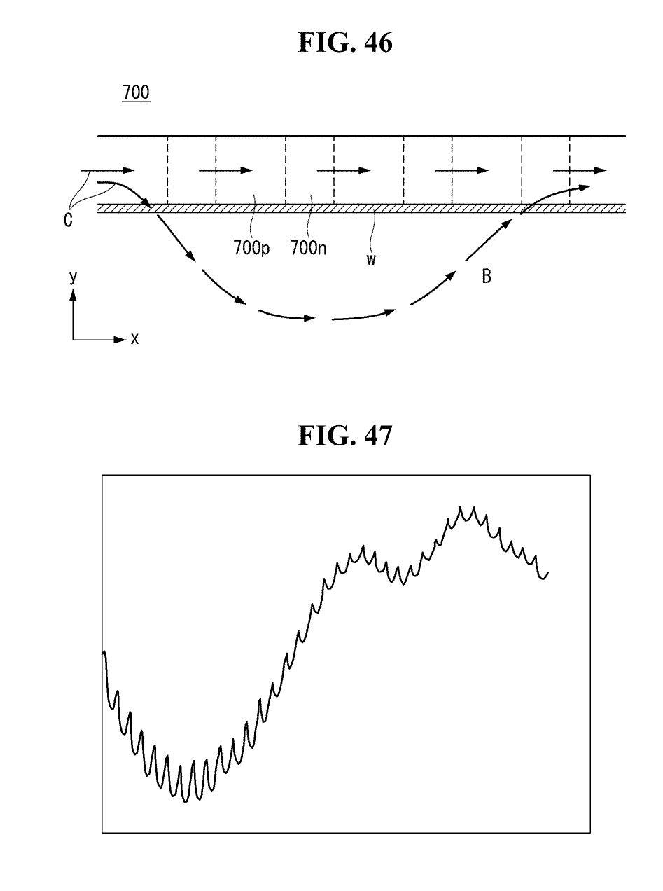

FIG. 46 illustrates another example of a cross section of an electrode according to an embodiment of the disclosure. More specifically, FIG. 46 illustrates an example of a cross-section of, for example, line A-A′ of FIG. 12. Further, FIG. 46 illustrates the electrode 700, a current C, a current flow area 700p, a non-current flow area 700n, a user's body B, and water W.

When a current flows in the electrode 700 in which the non-current flow area 700n is formed, an electrical resistance of the electrode 700 having the non-current flow area 700n may be greater than an electrical resistance of the electrode 700 not having the non-current flow area 700n. In other words, when a current flows in the electrode 700 in which the current flow area 700p and the non-current flow area 700n are formed, an electrical resistance of the electrode 700 having the current flow area 700p and the non-current flow area 700n may be greater than an electrical resistance of the electrode 700 having only the current flow area 700p. Hence, a temperature of the electrode 700 may increase. In embodiments disclosed herein, the resistance may mean a resistance of the x-axis direction.

Further, when the temperature of the electrode 700 increases, a temperature of a body part B of the user contacting the electrode 700 may increase. When a temperature of a user's skin increases, perspiration may be formed on the body part B of the user contacting the electrode 700. Namely, water W may be filled between the electrode 700 and the body part B of the user contacting the electrode 700. Hence, an electrical resistance between the user and the electrode 700 may decrease. In embodiments disclosed herein, the resistance may mean a resistance of the y-axis direction. In other words, an electrical resistance between the electrode 700 and the body part B of the user can decrease, and thus a current can flow smoothly between the electrode 700 and the body part B of the user.

FIGS. 47 to 50 illustrate examples of a biometric signal measured using an electrode.

More specifically, FIG. 47 illustrates an example of a biometric signal measured in a state where there is no movement of the user and the contact between the electrode and a body part of the user is performed well. In a graph of FIG. 47, peak values are displayed and can evaluate the biometric signal.

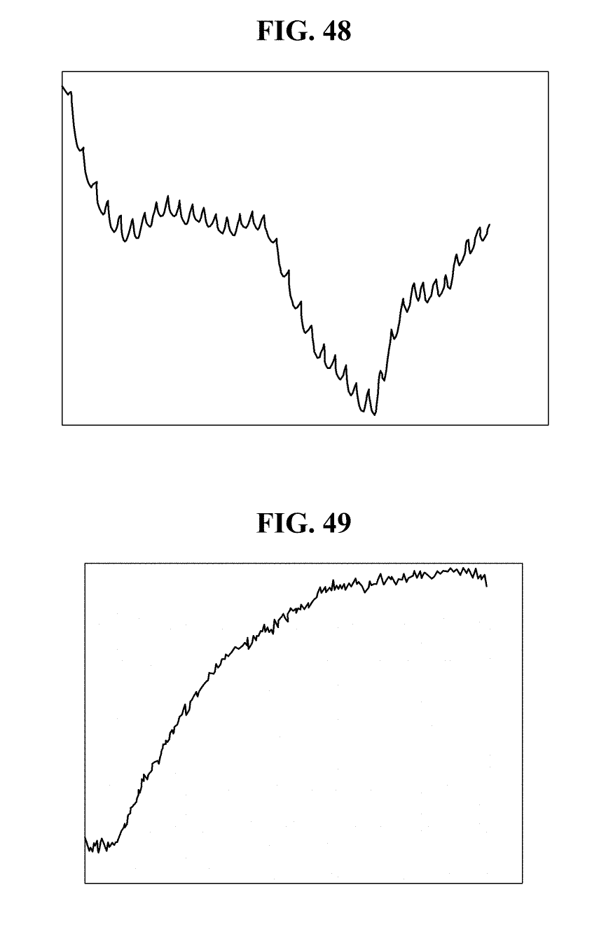

More specifically, FIG. 48 illustrates an example of a biometric signal measured in a state where there is no movement of the user and the contact between the electrode and a body part of the user is not performed well. In a graph of FIG. 48, peak values are displayed and can evaluate the biometric signal.

More specifically, FIG. 49 illustrates an example of a biometric signal measured in a state where there is a movement of the user and the contact between the electrode and a body part of the user is not performed well. In a graph of FIG. 49, peak values are not displayed and cannot evaluate the biometric signal.

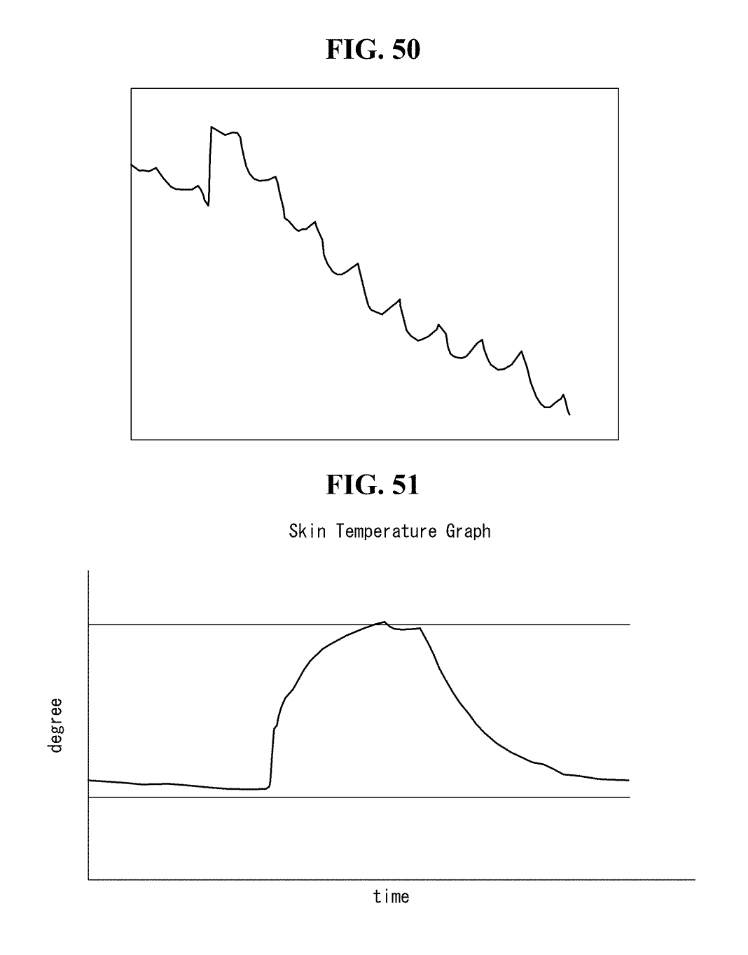

More specifically, FIG. 50 illustrates an example of a biometric signal measured in a state where there is a movement of the user and the contact between the electrode and a body part of the user is performed well. In a graph of FIG. 50, peak values are displayed and can evaluate the biometric signal.

Accordingly, obtaining an evaluable biometric signal may vary depending on a degree of the contact between the electrode and the body part of the user.

FIG. 51 illustrates an example of a skin temperature measured in a state where a user's skin contacts an electrode according to an embodiment of the disclosure. In a graph of a skin temperature illustrated in FIG. 51, a horizontal axis represents time, and a vertical axis represents a temperature. The skin temperature of the user can be accurately measured through the graph of the skin temperature. In embodiments disclosed herein, the skin temperature measured may have an error margin of about 0.2 degrees Celsius.

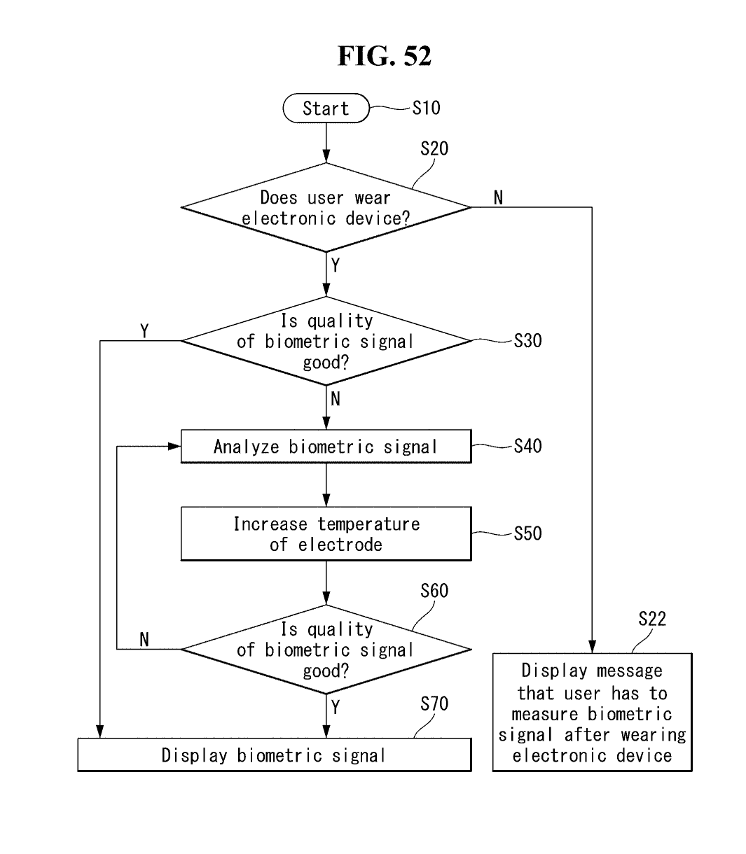

FIG. 52 illustrates an example of a method for controlling an electronic device according to an embodiment of the disclosure.

A user may intend to acquire his/her biometric signal using an electronic device in S10. In this instance, the electronic device may detect whether or not the user wears the electronic device in S20. When the user intends to acquire his/her biometric signal without wearing the electronic device, the electronic device may display a message that the user has to measure his/her biometric signal after wearing the electronic device in S22. When the user wears the electronic device, the electronic device may acquire his/her biometric signal. The electronic device may determine quality of the acquired biometric signal in S30. If the quality of the acquired biometric signal is good, the electronic device may display the biometric signal in S70. On the other hand, if the quality of the acquired biometric signal is not good, the electronic device may analyze the acquired biometric signal in S40. The analysis of the biometric signal may indicate whether or not the biometric signal is suitable for displaying biometric information. As a result of the analysis, when the biological signal is not suitable, a voltage applied to the electrode 700 of the electronic device may be increased. Hence, a temperature of the electrode 700 may increase in S50. The electronic device may determine whether or not the quality of the biometric signal acquired again after increasing the temperature of the electrode 700 is good in S60. If the quality of the again acquired biometric signal is good, the electronic device may display the biometric signal in S70. If the quality of the again acquired biometric signal is not good, the electronic device may again analyze the biometric signal in S40.

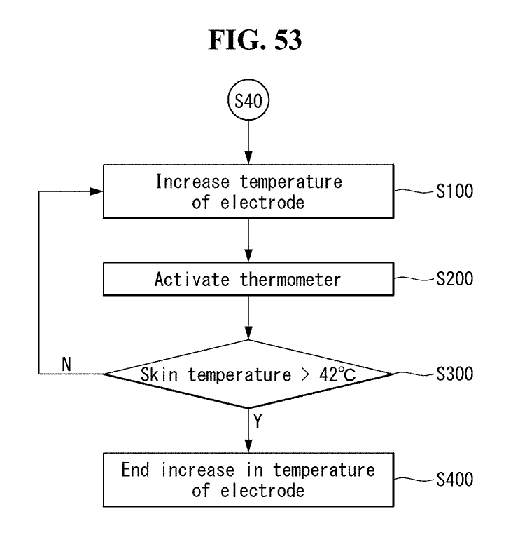

FIG. 53 illustrates another example of a method for controlling an electronic device according to an embodiment of the disclosure.

After the electronic device analyzes the biometric signal acquired through the electrode 700, the electronic device may increase the voltage applied to the electrode 700 and thus increase the temperature of the electrode 700 in S100. When the temperature of the electrode 700 increases by increasing the voltage applied to the electrode 700, the electronic device may activate a thermometer in S200. The electronic device may measure a skin temperature of the user through the activated thermometer and determine whether or not the skin temperature is maintained at an appropriate level. For example, the electronic device may determine whether or not the skin temperature exceeds 42 degrees Celsius in S300. If the skin temperature of the user does not exceed 42 degrees Celsius, the electronic device may maintain a state where the temperature of the electrode 700 is increased in S100. On the other hand, if the skin temperature of the user exceeds 42 degrees Celsius, the electronic device may reduce the voltage applied to the electrode 700 and end an increase in the temperature of the electrode 700 in S400. Hence, the electronic device can prevent the skin of the user from being burned by the heat generation of the electrode 700.

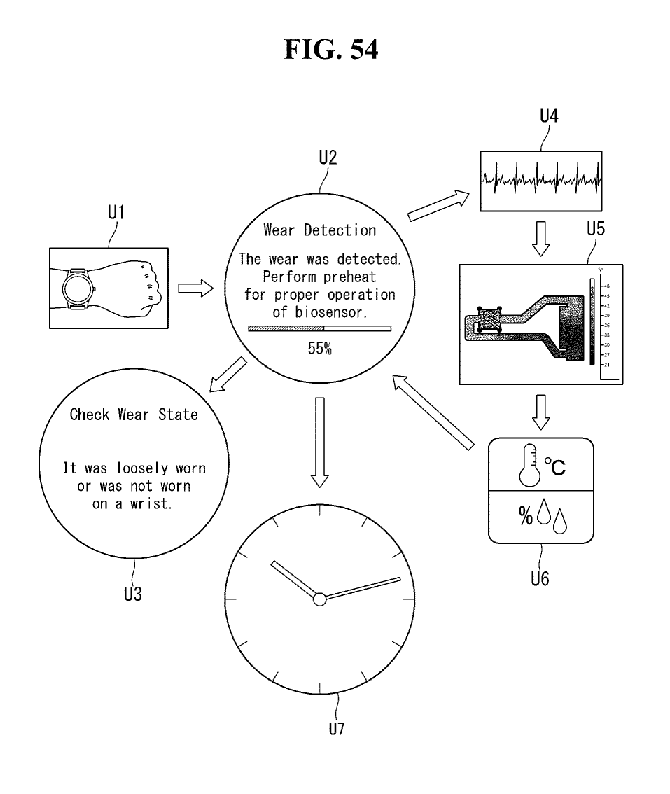

FIG. 54 illustrates an example of a method for operating an electronic device according to an embodiment of the disclosure.

When the user wears the electronic device in U1, the electronic device may detect wear of the electronic device and prepare for acquiring a biometric signal in U2. In this instance, depending on a state where the user wears the electronic device, for example, when the user wears the electronic device loosely or contactlessly, the electronic device may display a message that the user has to wear the electronic device more preferably in U3. The electronic device may acquire the biometric signal of the user and measure quality of the biometric signal in U4. If the quality of the biometric signal is good, the electronic device may activate the display unit in U7. On the other hand, if the quality of the biometric signal is not good, the electronic device may increase a temperature of the electrode 700 in U5. The electronic device may check a temperature and a humidity of the user's skin in accordance with an increase in the temperature of the electrode 700 in U6. This can prevent low temperature burns from occurring on the user's skin. When the quality of the biometric signal acquired in a state where the temperature and the humidity of the electrode 700 are increased is good, the electronic device may activate the display unit in U7. On the other hand, if the quality of the biometric signal is not good, the electronic device may increase the temperature of the electrode 700 in U5.

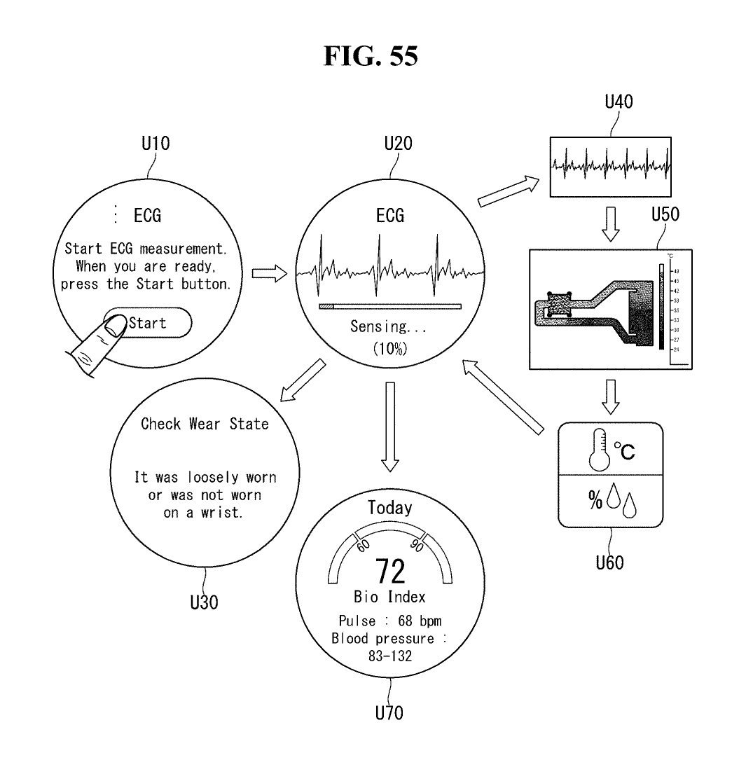

FIG. 55 illustrates another example of a method for operating an electronic device according to an embodiment of the disclosure.

When the user intends to measure his/her biometric signal using the electronic device in U10, the electronic device may prepare for acquiring the biometric signal in U20. In this instance, depending on a state where the user wears the electronic device, for example, when the user wears the electronic device loosely or contactlessly or when quality of the acquired biometric signal is remarkably low, the electronic device may display a message that the user has to wear the electronic device more preferably in U30. The electronic device may acquire the biometric signal of the user and measure the quality of the biometric signal in U40. If the quality of the biometric signal is good, the electronic device may activate the display unit and display biometric information in U70. On the other hand, if the quality of the biometric signal is not good, the electronic device may increase a temperature of the electrode 700 in U50. The electronic device may check a temperature and a humidity of the user's skin in accordance with an increase in the temperature of the electrode 700 in U60. This can prevent low temperature burns from occurring on the user's skin. When the quality of the biometric signal acquired in a state where the temperature and the humidity of the electrode 700 are increased is good, the electronic device may activate the display unit and display biometric information in U70. On the other hand, if the quality of the biometric signal is not good, the electronic device may increase the temperature of the electrode 700 in U50.

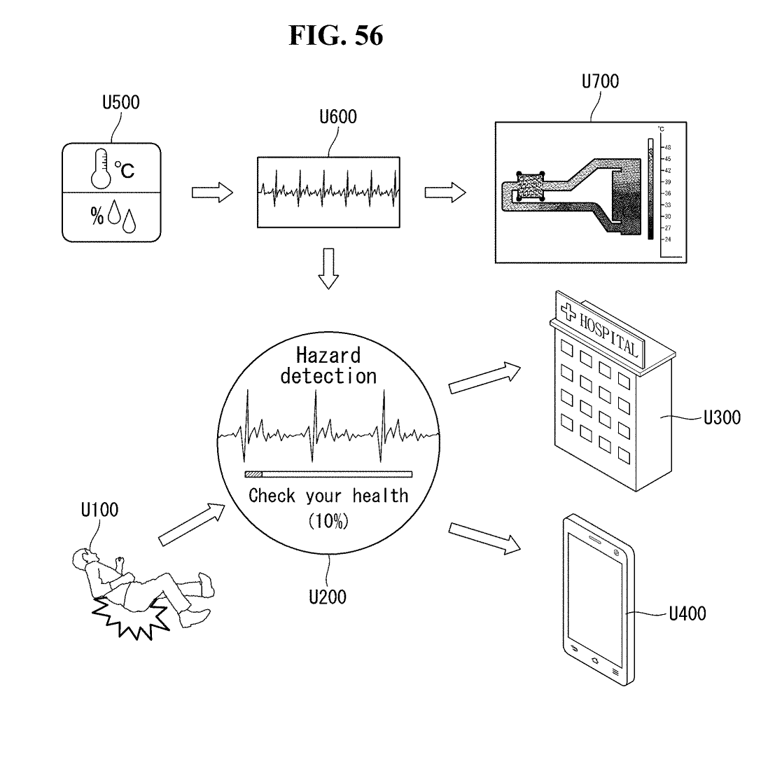

FIG. 56 illustrates yet another example of a method for operating an electronic device according to an embodiment of the disclosure.

The user wearing the electronic device may fall into a dangerous state in U100. For example, the dangerous state may include falls, gas poisoning, and the like. In this instance, the electronic device may measure a state of the user in U200. Depending on the state of the user, the electronic device may notify a nearby hospital of an emergency situation of the user in U300. Alternatively, the electronic device may notify previously set contacts of the emergency situation in U400. The previously set contacts may include emergency centers, relatives, and the like.

However, there may occur a problem that the electronic device cannot properly measure the state of the user. To this end, the electronic device may adjust the temperature of the electrode 700 in order to obtain a suitable biometric signal. When the electronic device constantly measures the temperature or the humidity of the user's skin and senses changes in the temperature or the humidity of the user's skin in U500, the electronic device may measure the quality of the biometric signal acquired through the electrode 700 in U600. In this instance, when the quality of the biometric signal is lowered, the electronic device may increase the voltage applied to the electrode 700 and thus increase the temperature of the electrode 700 and a temperature of a body part of the user contacting the electrode 700 in U700. Hence, the electronic device can immediately measure the biometric signal of the user without a time delay in emergency situations.

The foregoing embodiments are merely examples and are not to be considered as limiting the present disclosure. The present teachings can be readily applied to other types of methods and apparatuses. The features, structures, methods, and other characteristics of the embodiments described herein may be combined in various ways to obtain additional and/or alternative embodiments.

Although embodiments have been described with reference to a number of illustrative embodiments thereof, it should be understood that numerous other modifications and embodiments can be devised by those skilled in the art that will fall within the scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

1. An electronic device comprising:
a main body having a back surface contacting a body part of a user;
a band portion extended from one side or both sides of the main body, the band portion having a back surface contacting the body part of the user;
an electrode positioned on the back surface of the main body or the back surface of the band portion, the electrode having a current flow area and a non-current flow area; and
a controller mounted inside the main body or the band portion, the controller configured to adjust a voltage applied to the electrode in response to a biometric signal of the user acquired through the electrode.

2. The electronic device of claim 1, further comprising a driver configured to adjust the voltage applied to the electrode by the controller.

3. The electronic device of claim 1, wherein the non-current flow area is formed by an empty vacancy.

4. The electronic device of claim 1, wherein the current flow area has conductivity, and the non-current flow area has non-conductivity.

5. The electronic device of claim 1, wherein the electrode has a plurality of rows and a plurality of columns, and
wherein the non-current flow area is formed at intersections of the plurality of rows and the plurality of columns.

6. The electronic device of claim 1, wherein the electrode has a plurality of rows and a plurality of columns, and
wherein the current flow area is formed at intersections of the plurality of rows and the plurality of columns.

7. The electronic device of claim 6, wherein the electrode has a conductive path connecting the intersections to one another.

8. The electronic device of claim 1, wherein the electrode has a plurality of rows and a plurality of columns, and
wherein the non-current flow area is formed at intersections of odd-numbered rows of the plurality of rows and odd-numbered columns of the plurality of columns.

9. The electronic device of claim 1, wherein the electrode has a plurality of rows and a plurality of columns, and
wherein the non-current flow area is formed at intersections of even-numbered rows of the plurality of rows and even-numbered columns of the plurality of columns.

10. The electronic device of claim 1, wherein the electrode has a plurality of rows and a plurality of columns, and
wherein the current flow area is formed at intersections of odd-numbered rows of the plurality of rows and odd-numbered columns of the plurality of columns.

11. The electronic device of claim 1, wherein the electrode has a plurality of rows and a plurality of columns, and
wherein the current flow area is formed at intersections of even-numbered rows of the plurality of rows and even-numbered columns of the plurality of columns.

12. The electronic device of claim 10, wherein the electrode has a conductive path connecting the intersections to one another.

13. The electronic device of claim 1, wherein the electrode includes a heating element, and
wherein the controller adjusts a temperature of the heating element.

14. The electronic device of claim 1, wherein the band portion is extended from both sides of the main body to wrap the body part of the user,
wherein the electrode includes a plurality of electrodes,
wherein at least one of the plurality of electrodes is positioned on the back surface of the band portion extended from one side of the main body, and
wherein at least another of the plurality of electrodes is positioned on the back surface of the band portion extended from the other side of the main body.

15. The electronic device of claim 1, further comprising a sensor configured to measure a skin temperature of the user,
wherein the controller adjusts the voltage applied to the electrode depending on the skin temperature of the user obtained by the sensor.

16. The electronic device of claim 15, wherein the controller reduces the voltage applied to the electrode when the skin temperature of the user exceeds a predetermined temperature.

17. A method for controlling an electronic device comprising:
measuring a quality of a biometric signal of a user acquired by an electrode;
analyzing whether or not biometric information of the user is obtained from the biometric signal;
increasing a voltage applied to the electrode according to the analysis result; and
measuring a quality of a biometric signal of the user acquired by the electrode of which the voltage is increased.

18. The method of claim 17, further comprising, when the quality of the biometric signal is good, displaying the biometric information of the user.

19. The method of claim 17, further comprising:
measuring a skin temperature of the user when the voltage applied to the electrode is increased; and
reducing the voltage applied to the electrode when the skin temperature of the user exceeds a predetermined level.

20. The method of claim 17, further comprising, when the quality of the biometric signal is low, displaying a notification of wear of the electronic device.
